SEQUENCE LISTING (39 sequences)

SEQ ID NO: 1 - DNA, 60 bp, Artificial Sequence
  synthetic
atgcgtgagaatttacgttatgtaattcagtatcaccgctcaagttagtataaaaaagct

SEQ ID NO: 2 - DNA, 59 bp, Artificial Sequence
  synthetic
ttatttcactggctcaaccggtaaatctgcccgcgtgaagcctgcttttttatactaag

SEQ ID NO: 3 - DNA, 20 bp, Artificial Sequence
  synthetic
attagcatgcaggcggctac

SEQ ID NO: 4 - DNA, 18 bp, Artificial Sequence
  synthetic
tgctggaaaggccgcgaa

SEQ ID NO: 5 - DNA, 29 bp, Artificial Sequence
  synthetic
cgcagatcttgaagcctgcttttttatac

SEQ ID NO: 6 - DNA, 24 bp, Artificial Sequence
  synthetic
cgctcaagttagtataaaaaagct

SEQ ID NO: 7 - DNA, 25 bp, Artificial Sequence
  synthetic
cgcagatctcgagtccctatcagtg

SEQ ID NO: 8 - DNA, 20 bp, Artificial Sequence
  synthetic
aatttctcctctttccatgg

SEQ ID NO: 9 - DNA, 59 bp, Artificial Sequence
  synthetic
ccctgccacaatggcccgttagcaacgtcgaataacgctcaagttagtataaaaaagct

SEQ ID NO: 10 - DNA, 59 bp, Artificial Sequence
  synthetic
gtgatactgaattacataacgtaaattctcacgcatggtacctttctcctctttaatga

SEQ ID NO: 11 - DNA, 20 bp, Artificial Sequence
  synthetic
attagcatgcaggcggctac

SEQ ID NO: 12 - DNA, 21 bp, Artificial Sequence
  synthetic
gcaaaagaggctgatttggct

SEQ ID NO: 13 - DNA, 22 bp, Artificial Sequence
  synthetic
ttccattgggatgtagatgctg

SEQ ID NO: 14 - DNA, 24 bp, Artificial Sequence
  synthetic
ggtaaaagagtcaagggaagaacc

SEQ ID NO: 15 - DNA, 25 bp, Artificial Sequence
  synthetic
acgctttgacttctctcacttcggt

SEQ ID NO: 16 - DNA, 25 bp, Artificial Sequence
  synthetic
aaacatttgctcttgatgtcctgga

SEQ ID NO: 17 - DNA, 23 bp, Artificial Sequence
  synthetic
ctttgcaacctgtacatgccttg

SEQ ID NO: 18 - DNA, 25 bp, Artificial Sequence
  synthetic
catcgtagtagttgatcggatggac

SEQ ID NO: 19 - DNA, 51 bp, Artificial Sequence
  synthetic
ggggacaagtttgtacaaaaaagcaggctaagggggagaacacgatgaatg

SEQ ID NO: 20 - DNA, 50 bp, Artificial Sequence
  synthetic
ggggaccactttgtacaagaaagctgggtgtgccgaccaaagttcaggac

SEQ ID NO: 21 - protein, 297 aa, Homo sapiens
MASPQLCRALVSAQWVAEALRAPRAGQPLQLLDASWYLPKLGRDARREFEERHIPGAAFFDIDQCSDRTSPYDHMLPGAEHFAEYAGRLGVGAATHVVIYDASDQGLYSAPRVWWMFRAFGHHAVSLLDGGLRHWLRQNLPLSSGKSQPAPAEFRAQLDPAFIKTYEDIKENLESRRFQVVDSRATGRFRGTEPEPRDGIEPGHIPGTVNIPFTDFLSQEGLEKSPEEIRHLFQEKKVDLSKPLVATCGSGVTACHVALGAYLCGKPDVPIYDGSWVEWYMRARPEDVISEGRGKTH

SEQ ID NO: 22 - protein, 281 aa, Escherichia coli
MSTTWFVGADWLAEHIDDPEIQIIDARMASPGQEDRNVAQEYLNGHIPGAVFFDIEALSDHTSPLPHMLPRPETFAVAMRELGVNQDKHLIVYDEGNLFSAPRAWWMLRTFGVEKVSILGGGLAGWQRDDLLLEEGAVELPEGEFNAAFNPEAVVKVTDVLLASHENTAQIIDARPAARFNAEVDEPRPGLRRGHIPGALNVPWTELVREGELKTTDELDAIFFGRGVSYDKPIIVSCGSGVTAAVVLLALATLDVPNVKLYDGAWSEWGARADLPVEPVK

SEQ ID NO: 23 - protein, 284 aa, Yersinia pestis
MNSDFLVTPQWLAAHANDANIVILDARMSPPGVVPKRNIQAEFEQGHIPGAVYFDIDAIADHSTGLPHMLPSPQLFSEMAGQLGITEQHTVVIYDEGNLFSAPRVWWTFRTFGAKNVRILASGLSGWQQAGYKLESGPAHPTPQTFNVTFNAAAVSSVDEVLAVLGNNEVQILDARPSARYRAQEPEPRPGLRLGRIPGSINIPWGTMVENGHLKSPQALAEIFAAQGVDLTKPIITSCGSGVTAAVVVLGLAAVNARSVSLYDGSWAEWGASNSLPIDATPLA

SEQ ID NO: 24 - protein, 280 aa, Salmonella typhimurium
MTTAFFVAADWLAEHIDDPEIQILDARMAPPGQEHRDMAGEYRAGHIPGALFFDIEALSDRASPLPHMMPRPEAFAVAMRELGVRQDKHLVIYDEGNLFSAPRAWWMLRTFGAEKVSILAGGLAGWQRDEWLLREGEEAHEGGEFEAKFAPQAVVRLTDVLLASHEKTAQIVDARPAARFNAQADEPRPGLRRGHIPGALNVPWTELVYEGELKTTDELNEVFFSHGVSFDRPIIASCGSGVTAAVVVLALATLDVPDVTLYDGAWSEWGARTDLPVEPA

SEQ ID NO: 25 - protein, 284 aa, Brucella suis
MTEKSAFVVSRDWLKERLHKPGLAIVDASWYLPAAGRNGQEEYEKAHIPGAVFFDQDKIADKESGLPHTLPSPEFFAQQVGTLGITADETVVVYDGPGMFSAPRVWWMFRVMGVKNVYVLDGGFDGWKKAGYPVTDEVTKIAATFFKPSFNKDAVVDFQEMRKIVDEKRSQIADARGAGRFTGRDAEPRAEMRSGHMPGARNVPVTTLSENGELKDLESLRRIFDEAGIDLSGPVVTSCGSGVTAAVITLALTSLGHKDNRLYDGSWSEWGSRQDTPVVTGEAE

SEQ ID NO: 26 - protein, 289 aa, Erwinia pyrifoliae
MSASSSDLPVSHERFVSADWLANHLNDSSITLIDARMLPPGNDTRDIHAEYRAAHLPGAVFFDIENLSDHSTDLPHMMPTCENFARAMGELGIDNQQHLVIYDEGNLFSAPRAWWMLHTFGATSISILSGGLAGWKAQNLPLEQGYVTRKPVTFHATLDENAIRSRDDVLSISRDKSEQIVDARPASRFHAEVDEPRPGLHRGHIPGSLNVPWTDLVNNGALKPNAELATILHKHGVDFTRPIVASCGSGVTASVVVLALTQLNVPNVTLYDGSWSDWGSRDDVPIARD

SEQ ID NO: 27 - protein, 278 aa, Photorhabdus luminescens laumondii
MNNAYFVTPQWLKDHLDDKNLVILDATAPPPPQQIDCHKLWLNTHIPGAQFLDLDKIANHQSGLPHMLPDPQTFSQAVGAMGISENHLVVIYDQGNMFSAPRAWWTFKIFGSHNVRILDGGLQGWQQAGFPTASGEVKRNPQIFNTDFNADKVKSLEQILGALNDQQIQIVDARATDRFQAKAPEPRPGLRMGHIPGSKNIPWTMLVENGHFKSETEITDIFHKQGVDLNKPVITSCGSGMTAAVLVLGLDIIGKKDVYLYDGSWAEWGADEALPLEK

SEQ ID NO: 28 - protein, 286 aa, Rhodopseudomonas palustris
MTAHTDPLVSTDWLAERLGDPSVKIIDASFKMPGVLPLPADDYLAAHIPGAVFFDVDAVSDHASSLPHMYPSADQFARDVEALGISSGDTVVAYDAGGWVAAPRAWWMFLSFGHANIRILDGGLKKWVAEGLPTEAGKPTIAPGRFSAKLDPSFIRSRDQLVANLDSGAEQVIDARAAPRFEGSVAEPRPGLRAGHIPGSRNLPYNELFDAATGTMKPLAELRQAFERAGLDLGRPVVTSCGSGVSAAVLTLALYRLGVRGSALYDGSWSEWGLQDGPPVATGPAA

SEQ ID NO: 29 - protein, 292 aa, Nitrobacter winogradskyi
MSLMMDIAMNPTLDDPLVSTEWLAAHLGEVKAIDASFKMPGVLPLAVDDFYAAHIPGAVFFDVDAVSDRASPLPHMYPDAAQFGRDVGALGISSKDTVVVYDNGGWLAGPRAWWMFLSFGHAGVRVLDGGLKKWRAEGRPVESGKVSPEPGHFTATFDPLFVRDKAQLVSNLSSCREQLVDARAAARFTGAVMEPRQGLRSGHIPGSRNLSYAELFDPGTGVMKPLDDIRAAFSRAGVDLAKPVVTTCGSGVSAAVLTLALYRLGARGSALYDGSWSEWGLVEGPPIATGPA

SEQ ID NO: 30 - protein, 281 aa, Shigella boydii
MSTTWFVGADWLAEHIDDPEIQIIDARMASPGQEDRNVAQEYLNGHIPGAVFFDIEALSDHTSPLPHMLPRPKTFAVAMRELGVNQDKHLIVYDEGNLFSAPRAWWMLRTFGVEKVSILGGGLAGWQRDDLLLEEGAVELPEGEFNAAFNPEAVVKVTDVLLASHENTAQIIDARPAARFNAEVDEPRPGLRRGHIPGALNVPWTELVREGELKTTDELDAIFFGRGVSYDKPIIVSCGSGVTAAVVLLALATLDVPNVKLYDGAWSEWGARADLPVEPVK

SEQ ID NO: 31 - protein, 287 aa, Jannaschia sp. CCS1
MAEVITGDDPQTLVSTDWLAAHFNDPDLRIIDASYYLAEMNRDAKAEYDAGHIPGARFFDIDDISDARSELPHMVAPVEKFMSRMRAMGVGDGHQVVVYDGRGVFSAARVWWNFRLMGKTDVAVLDGGLPKWVAEGRPVEDMPPIIRDRHMTVQRQAHLVKDVTQVASASKLGDWQIVDARAPARFRGEEPETRPGLRAGRIPNSKNVHYASLFKPDGTMKEGDALRAAFEAGGVDLDKRIITTCGSGVTAAILMLGLTRLGHQDVSLYDGSWSEWGQFEQLKVETG

SEQ ID NO: 32 - protein, 315 aa, Homo sapiens
LKKIGDTPMVRINKIGKKFGLKCELLAKCEFFNAGGSVKDRISLRMIEDAERDGTLKPGDTIIEPTSGNTGIGLALAAAVRGYRCIIVMPEKMSSEKVDVLRALGAEIVRTPTNARFDSPESHVGVAWRLKNEIPNSHILDQYRNASNPLAHYDTTADEILQQCDGKLDMLVASVGTGGTITGIARKLKEKCPGCRIIGVDPEGSILAEPEELNQTEQTTYEVEGIGYDFIPTVLDRTVVDKWFKSNDEEAFTFARMLIAQEGLLCGGSAGSTVAVAVKAAQELQEGQRCVVILPDSVRNYMTKFLSDRWMLQKG

SEQ ID NO: 33 - protein, 300 aa, Bacillus anthracis
HELIGHTPIVEITRFSLPEGVRLFAKLEFYNPGGSVKDRLGRELIEDALEKGLVTEGGTIIEPTAGNTGIGLALAALQHDLRVIVCVPEKFSIEKQELMKALGATVVHTPTEQGMTGAIAKAKELVNEIPNSYSPSQFANEANPRAYFKTLGPELWSALNGEINIFVAGAGTGGTFMGTASYLKEKNIDIKTVIVEPEGSILNGGKAGSHETEGIGLEFIPPFLKTSYFDEIHTISDRNAFLRVKELAQKEGLLVGSSSGAAFHASLLEAEKAAPGTNIVTIFPDSSERYLSKDIYKGWE

SEQ ID NO: 34 - protein, 313 aa, Pseudomonas aeruginosa
LDLIGNTPLVRVTRFDTGPCTLYLKLESQNPGGSIKDRIGVAMIEAAERDGRLRPGGTIVEATAGNTGLGLALVGRAKGYRVVLVVPDKMSTEKVLHLRAMGAEVHITRSDVGKGHPEYYQDVAARLAQDIPGAFFADQFNNPANPLAHECGTGPELWAQTGHDLDAIVVGVGSSGTLTGLTRFFQKVQPELEMVLADPEGSIMAEYSRSGTLGTPGSWAVEGIGEDFVPAIADLSSVRHAYSISDEESFAMARELLRVEGIPGGSSTGTLLAAALRFCREQKEPKRVVSFVCDTGTRYLSKIYNDQWMTDQG

SEQ ID NO: 35 - protein, 298 aa, Staphylococcus aureus
YDLIGNTPLVLLEHYSDDKVKIYAKLEQWNPGGSVKDRLGKYLVEKAIQEGRVRAGQTIVEATAGNTGIGLAIAANRHHLKCKIFAPYGFSEEKINIMIALGADVSRTSQSEGMHGAQLAARSYAEKYGAVYMNQFESEHNPDTYFHTLGPELTSALQQIDYFVAGIGSGGTFTGTARYLKQHHVQCYAVEPEGSVLNGGPAHAHDTEGIGSEKWPIFLERRLVDGIFTIKDQDAFRNVKSLAINEGLLVGSSSGAALQGALNLKAQLSEGTIVVVFPDGSDRYMSKQIFNYEENDYE

SEQ ID NO: 36 - protein, 397 aa, Homo sapiens
MQEKDASSQGFLPHFQHFATQAIHVGQDPEQWTSRAVVPPISLSTTFKQGAPGQHSGFEYSRSGNPTRNCLEKAVAALDGAKYCLAFASGLAATVTITHLLKAGDQIICMDDVYGGTNRYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMEKHFKNGMAVAQFLESNPWVEKVIYPGLPSHPQHELVKRQCTGCTGMVTFYIKGTLQHAEIFLKNLKLFTLAESLGGFESLAELPAIMTHASVLKNDRDVLGISDTLIRLSVGLEDEEDLLEDLDQALKAA

SEQ ID NO: 37 - protein, 377 aa, Bacillus anthracis
MRAKTKLIHGIRIGEPSTGSVNVPIYQTSTYKQEAVGKHQGYEYSRTGNPTRAALEEMIAVLENGHAGFAFGSGMAAITATIMLFSKGDHVILTDDVYGGTYRVITKVLNRFGIEHTFVDTTNLEEVEEAIRPNTKAIYVETPTNPLLKITDIKKISTLAKEKGLLTIIDNTFMTPYWQSPISLGADIVLHSATKYLGGHSDVVAGLVVVNSPQLAEDLHFVQNSTGGILGPQDSFLLLRGLKTLGIRMEEHETNSRAIAEFLNNHPKVNKVYYPGLASHQNHELATEQANGFGAIISFDVDSEETLNKVLERLQYFTLAESLGAVESLISIPSQMTHASIPADRRKELGITDTLIRISVGIEDGEDLIEDLAQALA

SEQ ID NO: 38 - protein, 394 aa, Pseudomonas aeruginosa
MSQHDQHPDAPAQAFATRVIHAGQAPDPSTGAIMPPIYANSTYIQESPGVHKGLDYGRSHNPTRWALERCVADLEGGTQAFAFASGLAAISSVLELLDAGSHIVSGNDLYGGTFRLFERVRRRSAGHRFSFVDPTDLQAFEAALTPETRMVWVETPSNPLLRLTDLRAIAQLCRARGIISVADNTFASPYIQRPLELGFDVVVHSTTKYLNGHSDVIGGIAIVGDNPDLRERLGFLQNSVGAISGPFDAFLTLRGVKTLALRMERHCSNALALAQWLERQPQVARVYYPGLASHPQHELAKRQMRGFGGMISLDLRCDLAGARRFLENVRIFSLAESLGGVESLIEHPAIMTHASIPAETRADLGIGDSLIRLSVGVEALEDLQADLAQALAKI

SEQ ID NO: 39 - protein, 380 aa, Staphylococcus aureus
MNKKTKLIHGGHTTDDYTGAVTTPIYQTSTYLQDDIGDLRQGYEYSRTANPTRSSVESVIAALENGKHGFAFSSGVAAISAVVMLLDKGDHIILNSDVYGGTYRALTKVFTRFGIEVDFVDTTHTDSIVQAIRPTTKMLFIETPSNPLLRVTDIKKSAEIAKEHGLISVVDNTFMTPYYQNPLDLGIDIVLHSATKYLGGHSDVVAGLVATSDDKLAERLAFISNSTGGILGPQDSYLLVRGIKTLGLRMEQINRSVIEIIKMLQAHPAVQQVFHPSIESHLNHDVHMAQADGHTGVIAFEVKNTESAKQLIKATSYYTLAESLGAVESLISVPALMTHASIPADIRAKEGITDGLVRISVGIEDTEDLVDDLKQALDTL

(19) United States
(12) Patent Application Publication
(10) Pub. No.: US 2014/0066362 A1
(43) Pub. Date: Mar. 6, 2014

(54) METHOD FOR TREATING INFECTIONS BY TARGETING MICROBIAL H2S-PRODUCING ENZYMES

(71) Applicants: Evgeny A. Nudler, New York, NY (US); Konstantin Shatalin, Springfield, NJ (US)

(72) Inventors: Evgeny A. Nudler, New York, NY (US); Konstantin Shatalin, Springfield, NJ (US)

(73) Assignee: NEW YORK UNIVERSITY, New York, NY (US)

(21) Appl. No.: 13/983,149

(22) PCT Filed: Feb. 1, 2012
(86) PCT No.: PCT/US2012/023542

§ 371 Date: Nov. 15, 2013

Related U.S. Application Data
(60) Provisional application No. 61/438,524, filed on Feb. 1, 2011.

(51) Int. Cl. A61K 38/16; A61K 45/06

(52) U.S. Cl. CPC A61K 38/16; A61K 45/06
USPC 514/2.3

(57) ABSTRACT

The invention provides materials and methods for treating infections by reducing endogenous microbial H₂S levels.

### CROSS-REFERENCE TO RELATED APPLICATIONS

This application claims the benefit of U.S. Provisional Patent Application No. 61/438,524, filed Feb. 1, 2011, which is incorporated herein by reference in its entirety.

### STATEMENT AS TO FEDERALLY SPONSORED RESEARCH

This invention was made with government support under grant No. 5DP1OD000799, awarded by the National Institutes of Health. The government has certain rights in the invention.

### TECHNICAL FIELD OF THE INVENTION

The invention relates to materials and methods for treating infections, and more particularly to materials and methods for reducing endogenous microbial H₂S levels.

### BACKGROUND OF THE INVENTION

Despite the phenomenal success of antibiotics, infectious diseases remain the second leading cause of death worldwide. About two million Americans are infected in hospitals each year (with 90,000 deaths as a result), and more than half of these infections resist at least one antibiotic. For example, pathogens can alarmingly become fully resistant to last resort antibiotics, such as vancomycin. The emergence of multidrug-resistant bacteria has created a situation in which there are few or no options for treating certain infections. Natural antibiotics and their derivatives are intrinsically prone to become obsolete because of preexisting genes that render pathogens resistant to them. Bacterial species share these genes, thus rapidly spreading resistance from hospitals and farms to surrounding communities.

H₂S is a toxic gas that has been associated with beneficial functions in mammals, including vasorelaxation, cardioprotection, neurotransmission, and anti-inflammatory action in the gastrointestinal (GI) tract (1-5). The ability of H₂S to function as a signaling molecule parallels the action of another established gasotransmitter, nitric oxide (NO) (6-8). Endogenous NO was demonstrated to protect certain Gram(+) bacteria against antibiotics and oxidative stress (10-12).

Like NO, H₂S is produced enzymatically in various tissues (1, 4). Three H₂S-generating enzymes have been characterized in mammals: cystathionine β-synthase (CBS), cystathionine γ-lyase (CSE) and 3-mercaptopyruvate sulfurtransferase (3MST). CBS and CSE produce H₂S predominantly from L-cysteine (Cys). 3MST does so via the intermediate synthesis of 3-mercaptopyruvate by cysteine aminotranferase (CAT), which is inhibited by aspartate (Asp) competition for Cys on CAT (9) (FIG. 1A). In contrast to mammals, bacteria-derived H₂S has been known for centuries, but has been considered to be merely a byproduct of sulfur metabolism with no particular physiological function in non-sulfur microorganisms (it is used as energy source in purple and green sulfur bacteria). Further, little is known about the metabolic pathways involving H₂S in mesophilic bacteria. Analysis of bacterial genomes, however, revealed that most, if not all, possess orthologs of mammalian CBS, CSE, or 3MST (FIGS. 1A and 2A-C), suggesting an important cellular function(s) that preserved these genes throughout bacterial evolution.

### SUMMARY OF THE INVENTION

As indicated in the Background section, above, there is a great need in the art to develop new effective treatments for microbial infections and to fight antibiotic resistance.

The present invention addresses these and other needs by providing methods and compositions for treating infections and enhancing effectiveness of antibiotics by reducing endogenous microbial H₂S levels.

In one aspect, the invention provides a method for treating a subject having a microbial infection, said method comprising administering to said subject a therapeutically effective amount of at least one inhibitor of endogenous H₂S production by an organism causing the microbial infection. In one embodiment, the method further comprises administering a second antimicrobial compound (e.g., a quinolone, an acridine, a phenothiazine, an aminoglycoside, a macrolide, an amphenicol, a steroid, an ansamycin, an antifolate, a polymyxin, a glycopeptide, a cephalosporin, a lactam, or any combination thereof). In one specific embodiment, the second antimicrobial compound is selected from the group consisting of novobiocin, norfloxacin, nalidixic acid, oxolinic acid, acriflavine, 9-aminoacridine, proflavine, trifluoperazine, promethazine, chlorpromazine, streptomycin, apramycin, tylosin, oleandomycin, erythromycin, josamycin, spiramycin, troleandomycin, chloramphenicol, fusidic acid, rifamycin SV, trimethoprim, 2,4-diamino-6,7-diisopropylpteridine, polymyxin B, colistin, vancomycin, cefsulodin, cephalothin, cefoperazone, oxacillin, nafcillin, phenethicillin, penicillin G, cloxacillin, moxalactam, carbenicillin, and any combination thereof. In another specific embodiment, the second antimicrobial compound is selected from the compounds disclosed in Tables 1 and 2 (below) and any combination thereof. The inhibitor of endogenous H₂S production and the second antimicrobial compound can be administered simultaneously (within the same composition or in different compositions) or sequentially (with either the inhibitor of endogenous H₂S production or the second antimicrobial compound administered first).

In another aspect, the invention provides a method for enhancing efficacy of an antimicrobial treatment in a subject having a microbial infection, wherein said antimicrobial treatment comprises administering to the subject a first antimicrobial compound that becomes compromised by H₂S or natural products of H₂S metabolism in vivo, said method comprising co-administering said first compound with a therapeutically effective amount of a second compound which second compound is an inhibitor of endogenous H₂S production by an organism causing the microbial infection. The first antimicrobial compound that becomes compromised by H₂S or natural products of H₂S metabolism in vivo can be without limitation, e.g., a quinolone, an acridine, a phenothiazine, an aminoglycoside, a macrolide, an amphenicol, a steroid, an ansamycin, an antifolate, a polymyxin, a glycopeptide, a cephalosporin, a lactam, or any combination thereof. In one specific embodiment, the first antimicrobial compound that becomes compromised by H₂S or natural products of H₂S metabolism in vivo is selected from the group consisting of novobiocin, norfloxacin, nalidixic acid, oxolinic acid, acriflavine, 9-aminoacridine, proflavine, trifluoperazine, promethazine, chlorpromazine, streptomycin, apramycin, tylosin, oleandomycin, erythromycin, josamycin, spiramycin, troleandomycin, chloramphenicol, fusidic acid, rifamycin SV, trimethoprim, 2,4-diamino-6,7-diisopropylpteridine, polymyxin B, colistin, vancomycin, cefsulodin, cephalothin, cefoperazone, oxacillin, nafcillin, phenethicillin, penicillin G, cloxacillin, moxalactam, carbenicillin, and any combination thereof. In another specific embodiment, the first antimicrobial compound that becomes compromised by H₂S or natural products of H₂S metabolism in vivo is selected from the compounds disclosed in Tables 1 and 2 (below) and any combination thereof. The first antimicrobial compound that becomes compromised by H₂S or natural products of H₂S metabolism in vivo and the inhibitor of endogenous H₂S production can be administered simultaneously (within the same composition or in different compositions) or sequentially.

In yet another aspect, the invention provides a method for sensitizing a microbial pathogen to oxidative damage comprising administering to said pathogen an effective amount of at least one inhibitor of endogenous H₂S production by said pathogen. In one embodiment, the pathogen is in a subject and the inhibitor of endogenous H₂S production is administered to the subject.

In one embodiment, the inhibitor of endogenous H₂S production useful in the methods of the present invention inhibits an H₂S-generating enzyme within the organism causing the microbial infection. In one embodiment, the inhibitor of endogenous H₂S production inhibits microbial cystathionine β-synthase (CBS) or paralogs thereof. In a specific embodiment, such CBS inhibitor can be co-administered (simultaneously in the same or separate compositions or sequentially) with an inhibitor of microbial cystathionine γ-lyase (CSE) or paralogs thereof. In a separate embodiment, the inhibitor of endogenous H₂S production inhibits microbial 3-mercaptopyruvate sulfurtransferase (3MST) or paralogs thereof. In another embodiment, the inhibitor of endogenous H₂S production inhibits microbial cystathionine γ-lyase (CSE) or paralogs thereof.

In one embodiment, the inhibitor of endogenous H₂S production is amino-oxyacetate (AOAA). In another embodiment, the inhibitor of endogenous H₂S production is hydroxylamine. In yet another embodiment, the inhibitor of endogenous H₂S production is D,L-propargylglycine (PAG). In a further embodiment, the inhibitor of endogenous H₂S production is β-cyano-L-alanine. In another embodiment, the inhibitor of endogenous H₂S production is aspartate or a derivative thereof. In an additional embodiment, the inhibitor of endogenous H₂S production is homocysteine.

In one embodiment, the inhibitors of endogenous H₂S production useful in the methods of the present invention selectively inhibit H₂S-generating enzyme(s) within the organism causing the microbial infection, but not H₂S-generating enzyme(s) in the cells of the subject being treated. Such selective inhibitors can be identified using various screening methods known in the art, e.g., as described in International Application Publication No. WO 2011/130181.

The methods of the present invention are useful for treating infections caused by various microorganisms, including, e.g., bacteria, fungi and protozoa. Non-limiting examples of encompassed bacterial genera include, e.g., Bacillus, Brucella, Clostridium, Enterococcus, Escherichia, Francisella, Helicobacter, Klebsiella, Legionella, Listeria, Mycobacterium, Pseudomonas, Salmonella, Shigella, Staphylococcus, Streptococcus, Trypanasoma, Vibrio, and Yersinia. In one specific embodiment, bacteria are from a species selected from the group consisting of E. coli, S. aureus, B. anthracis, and P. aeruginosa.

Non-limiting examples of microbial infectious diseases which can be treated by the methods of the present invention include, e.g., pneumonia, bronchitis, diphtheria, pertussis (whooping cough), tetanus, endocarditis, sepsis, bacterial gastroenteritis, cholera, tuberculosis, gonorrhea, chlamydia, syphilis, bacterial meningitis, malaria; trachoma, leishmaniasis, chagas disease, trichomoniasis, lyme disease, and leprosy.

The methods of the present invention can be used to treat infections in various animals, including, e.g., mammals, birds and fish. In one specific embodiment, the methods of the present invention are used to treat infections in humans.

The methods of the present invention can further comprise administering an inhibitor of microbial endogenous nitric oxide (NO) production or a NO scavenger. Such inhibitor of microbial endogenous nitric oxide (NO) production or a NO scavenger can be administered simultaneously (within the same composition or in different compositions) or sequentially (in any order) with an inhibitor of endogenous H₂S production and/or the second antimicrobial compound. Non-limiting examples of useful inhibitors of endogenous NO production include, e.g., L-arginine, NG-monomethyl-L-arginine, NG-nitro-L-arginine methyl ester, NG-nitro-L-arginine, NG-amino-L-arginine, NG,NG-dimethylarginine (asymmetric dimethylarginine), L-thiocitrulline, S-methyl-L-thiocitrulline, diphenyleneiodonium chloride, 2-(4-carboxyphenyl)-4,4,5,5-tetramethylimidazoline-1-oxy 3-oxide, 7-nitroindazole, N(5)-(1-iminoethyl)-L-ornithine, aminoguanidine, canavanine, ebselen, S-methyl-L-citrulline, S-methylisourea, and 2-mercaptoethylguanidine. In one specific embodiment (wherein the microbial infection is caused by Gram (+) bacteria), the inhibitor of endogenous NO production is an iNOS-specific inhibitor. Non-limiting examples of useful NO scavengers include, e.g., non-heme iron-containing peptides, non-heme iron-containing proteins, porphyrins, metalloporphyrins, dithiocarbamates, dimercaptosuccinic acid, phenanthroline, desferrioxamine, pyridoxal isonicotinoyl hydrazone, 1,2-dimethyl-3hydroxypyrid-4-one, [+] 1,2-bis(3,5-dioxopiperazinelyl)propane, and 2-(4-carboxyphenyl)-4,5-dihydro-4,4,5,5-tetramethyl-1H-imidazolyl-1-oxy-3-oxide. In one specific embodiment, the NO scavenger is a perfluorocarbon emulsion.

In conjunction with the methods of the present invention, provided herein are various combination pharmaceutical compositions. In one embodiment, the invention provides a pharmaceutical composition comprising (i) an antimicrobial compound that becomes compromised by H₂S or natural products of H₂S metabolism in vivo and (n) an inhibitor of microbial endogenous H₂S production. In a specific embodiment, this composition further comprises (iii) an inhibitor of microbial endogenous nitric oxide (NO) production or a NO scavenger. In a separate embodiment, the invention provides a pharmaceutical composition comprising (i) an inhibitor of microbial endogenous H₂S production and (ii) an inhibitor of microbial endogenous nitric oxide (NO) production or a NO scavenger.

Unless otherwise defined, all technical and scientific terms used herein have the same meaning as commonly understood by one of ordinary skill in the art to which this invention pertains. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

FIG. 1A is a schematic representation of basic metabolic pathways of H₂S synthesis. CBS and CSE generate H₂S with pyruvate and ammonia directly from Cys. 3MST generates H₂S and pyruvate from 3-mercaptopyruvate (3MP), which is synthesized along with glutamate by CAT from Cys and α-ketoglutarate. The orthologs of corresponding mammalian genes are present in the majority of bacterial species whose genomes have been fully sequenced (FIG. 5). Chemicals that specifically inhibit CBS, CSE, and 3MST are indicated. A sample of clinically relevant pathogens carrying 3MST or CBS/CSE encoding genes is shown on the right.

FIG. 2A-C are sample sequence alignments of H₂S-producing enzymes. (A) 3MST from Homo sapiens (HS; SEQ ID NO: 21) and its orthologs from E. coli (EC; SEQ ID NO: 22), Y. pestis CO92 (YP; SEQ ID NO: 23), S. typhimurium LT2 (ST; SEQ ID NO: 24), B. suis 23445 (BS; SEQ ID NO: 25), E. pyrifoliae Ep1/96 (ER; SEQ ID NO: 26), P. luminescens laumondii TTO1 (PL; SEQ ID NO: 27), R. palustris CGA009 (RP; SEQ ID NO: 28), N. winogradskyi Nb-255 (NW; SEQ ID NO: 29), S. boydii Sb227 (SB; SEQ ID NO: 30), and Jannaschia sp. CCS1 (JS; SEQ ID NO: 31) (S9, S10). (B) CBS from Homo sapiens (HS; SEQ ID NO: 32) and their homologs from B. anthracis Sterne (BA; SEQ ID NO: 33), P. aeruginosa PA01 (PA; SEQ ID NO: 34) and S. aureus N315 (SA; SEQ ID NO: 35). (C) CSE from Homo sapiens (HS; SEQ ID NO: 36) and their homologs from B. anthracis Sterne (BA; SEQ ID NO: 37), P. aeruginosa PA01 (PA; SEQ ID NO: 38) and S. aureus N315 (SA; SEQ ID NO: 39). Conserved amino acids are highlighted. Active-site loop residues of HS 3MST and CBS are shown in bold (S11-13). Asterisks indicate key active-site residues of CSE (S14).

FIG. 3A-B show genome organization of 3MST and CBS/CSE in selected bacteria. (A) Localization of sseA (3MST) orthologs (black arrow block aligned in the middle) in selected genomes of pathogenic bacteria. Genes of same color are from the same ortholog group. Light grey—no COG assignment; white—pseudo gene. (B) CBS (black arrow block aligned in the middle) and CSE (white arrow block aligned in the middle) orthologs in B. anthacis, S. aureus and P. aeruginosa genomes.

FIG. 4A-B demonstrate that endogenous H₂S protects bacteria against antibiotic toxicity. (A) H₂S production by B. anthracis, S. aureus, P. aeruginosa, and E. coli depends on CBS/CSE and 3MST, respectively. Lead acetate-soaked paper strips show a PbS brown or black stain as a result of reaction with H₂S. Strips were affixed to the inner wall of a culture tube, above the level of the liquid culture of wt or mutant bacteria, for 18 hours. CBS/CSE and 3MST inhibitors PAG/AOAA (inh) and aspartate (Asp, 3.2 mM), respectively, were added as indicated. Numbers (%) show the relative decrease in H₂S production due to chemical or genetic inhibition of CBS/CSE and 3MST. pMST indicates the E. coli strain that expresses an extra copy of the 3MST gene under a strong pLtetO-1 promoter. (B) Cysteine (Cys) is a substrate for bacterial CBS/CSE and 3MST. Addition of Cys (25 mM for E. coli; 200 mM for other species) greatly stimulated. H₂S synthesis in wt, but not in CBS/CSE- or 3MST-deficient strains.

FIG. 4C is a Pb(Ac)₂ analysis of individual strains harboring knockouts.

FIG. 5 shows the results of Phenotype MicroArray. The growth curves for E. coli wt are shown in black, for ΔsseA in white and overlay in black. Data are shown as the means from two experiments. The relative values of growth inhibition (negative numbers) are presented in Table 1.

FIG. 6 shows growth of wt and mutant E. coli, B. anthracis, and P. aeruginosa in LB.

FIG. 7 demonstrates that H₂S suppresses antibiotic-mediated bacterial killing. Representative survival curves show the effect of CBS/CSE (B. anthracis) and 3MST (E. coli) deletions or CBS/CSE inhibition (S. aureus and P. aeruginosa) by PAG/AOAA (inh) on Gm-mediated (50 mg/ml) killing. Where indicated, NaHS (0.2 mM) was added before the antibiotic challenge. The percentage of surviving cells was determined by counting colony-forming units (CFU) and is shown as the mean±SD from three experiments.

FIG. 8A-C show that endogenous H₂S protects various bacteria against diverse antibiotics. (A) Representative optical density (OD) growth curves of E. coli (MG1655), B. anthracis (Sterne) or P. aeruginosa (PA14) (black squares) and their H₂S-deficient counterparts (gray circles) in the presence of spectinomycin (Sp, 60 μg/ml), nalidixic acid (NA, 2.5 μg/ml), erythromycin (Em, 1 μg/ml), or ampicillin (Amp, 125 μg/ml). Gray triangles show the growth of E. coli that overexpress 3MST. Cells were grown in triplicate at 37° C. with aeration using a Bioscreen C automated growth analysis system. The curves represent averaged values from three parallel experiments with a margin of error of less than 5%. (B) Inhibitors of CSE/CSB and 3MST increase antibiotic sensitivity. Panels show representative growth curves of methicillin-resistant S. aureus (MW2), E. coli, or P. aeruginosa (black squares) in the presence of chloramphenicol (Cm, 0.5 μg/ml), vancomycin (Van, 0.5 μg/ml), acriflavine (Acr, 8 μg/ml), or pyocyanin (Pyo, 50 μM). Inverted gray triangles and gray circles represent cellular growth in the presence of CSE/CSB and 3MST inhibitors (inh) with or without antibiotics, respectively. (C) Exogenous H₂S restores antibiotic resistance in H₂S-deficient bacteria. Panels show representative growth curves of wt E. coli or B. anthracis (black squares) or H₂S-deficient strains (gray circles) in the presence of gentamicin (Gm, 1 μg/ml), NA (2.5 μg/ml), cefuroxime (Cef, 20 μg/ml), and Pyo (50 μM). Gray triangles show growth of cells pretreated with the H₂S donor (NaHS) in the presence of the indicated antibiotic.

FIG. 9 shows that H₂S protects Gram(+) bacteria against chloramphenicol-mediated killing. Survival curves show the effect of CBS/CSE (B. anthracis and P. aeruginosa) and 3MST (E. coli) deletions or CBS/CSE inhibition (S. aureus) by AOAA/PAG (inh) on chloramphenicol (Cm, 50 μg/ml)-mediated killing. This “bacteriostatic” antibiotic kills Gram(+) bacteria (S. aureus and B. anthracis) within a relatively short time period, but not Gram(−) E. coli and P. aeruginosa. The data suggest that Cm, and perhaps other bacteriostatic antibiotics, exert oxidative damage in Gram(+) bacteria, against which H₂S provides protection, thereby improving growth in the presence of these antibiotics in liquid culture (FIG. 8B).

FIG. 10A-N show that H₂S protects against antibiotic-inflicted oxidative damage. (A) H₂S acts by diminishing reactive oxygen species (ROS)-mediated antibiotic toxicity. E. coli cells were pretreated with the iron chelator, 2,2′-dipyridyl (0.05 mM) or the ROS scavenger thiourea (15 mM) for 3 min, followed by treatment with Gm. Cells were grown in triplicate at 37° C. with aeration using a Bioscreen C automated growth analysis system. The curves represent averaged values from three parallel experiments with a margin of error of less than 5%. (B) Endogenous H₂S renders cells more resistant to NA in aerobic conditions, but fails to do so in anaerobic conditions. A paper disk saturated with 20 mg/ml NA was placed on wt or CBS/CSE-deficient B. anthracis lawns that were grown aerobically or anaerobically for the next 18 hours. Zone borders are marked with dashed lines. (C) Endogenous H₂S renders B. anthracis more resistant to pyocyanin in aerobic conditions, but fails to do so in anaerobic conditions. A paper disk saturated with 50 iM pyocyanin was placed on wt or CBS/SCE-def B. anthracis lawns that were grown aerobically or anaerobically for the next 20 hours. Zone borders are marked with dashed lines. (D) Endogenous H₂S renders bacteria resistant to hydrogen peroxide. Agar plates seeded with the indicated bacteria were incubated overnight with a filter paper disk saturated with 0.125 or 0.45 M H₂O₂ placed atop the bacterial lawn. CBS/CSE- or 3MST-deficient cells formed a clear 5- to 10-mm zone around the disk, whereas wt cells grew a complete lawn and so demonstrated strong H₂S-dependent resistance to hydrogen peroxide. (E) 3MST-deficent cells exhibit a much greater sensitivity to hydrogen peroxide than other cysteine desulfurase mutants. Overnight cultures of indicated E. coli strains were diluted with fresh LB 1:50 and grown to OD₆₀₀ ˜1. H₂O₂ was added to 2.5 mM for 10 min. Cell survival was determined by counting CFU and is shown as the mean±SD from three independent experiments. (F) Rapid protective effect of H₂S against oxidative stress. Wt E. coli cells were grown in LB to OD₆₀₀ ˜1.0, treated with NaHS (200 μM), or the iron chelator dipyridyl (0.5 mM), or both for 1 min, followed by the addition of H₂O₂ (2 mM) for 10 min. Cell survival was determined by counting CFU and is shown as the mean±SD from three independent experiments. (G) Inhibitors of CBS/CSE render S. aureus sensitive to hydrogen peroxide, whereas an H₂S donor reverses this sensitivity. The panel shows growth curves of methicillin-resistant S. aureus (MW2) pretreated with PAG and AOAA for 3 min, followed by the addition of H₂O₂ (0.5 mM) (circles). NaSH was added to 100 μM prior to challenge with H₂O₂ (triangles). (H) Pulsed-field gel analysis of chromosomal DSBs. Lane 1: 4.6 Mb linearized E. coli chromosomes (I-SceI); lanes 2 and 3: DNA from wt and DMST cells; lanes 6 to 8: DNA from wt, 3MST-deficient, and 3MST-overproducing cells after treatment with 10 mg/ml Amp; lanes 9 and 10: DNA from NaHS-treated cells after Amp treatment; and lane 11: concatemers from 0.05 to 1.0 Mb. “% linear” indicates the relative increase in linearized chromosomal DNA. The values are the average of three independent experiments (P<0.1). (I) H₂S protects against H₂O₂— and antibiotic-inflicted DNA damage. Sub-lethal amounts of H₂O₂ and ampicillin (Amp) inflicted greater chromosome damage in CBS/CSE-deficient B. anthracis and P. aeruginosa and 3MST-deficient E. coli cells than in wt cells. The integrity of chromosomal DNA was monitored by PCR. Representative agarose gels show chromosomal fragments amplified from equal amounts of genomic DNA isolated from wild type (wt) or mutant (Δ) cells. M, 1 kb DNA marker. “%” indicates the fraction of the full-length PCR products. Values are the averages from three experiments with a margin of error of less than 10%. (3) H₂S suppresses antibiotic-induced SOS response. Gentamicin (Gm, 20 μg/ml) induces fluorescence in recA′::gfp E. coli (wt). SOS caused by Gm was greater in Asp-treated cells, but fully suppressed by exogenous NaHS. The genotoxic agent mitomycin (MMC, 0.2 μg/ml) was used as a positive control. In each case, the basal level of fluorescence before induction was assigned a value of 1. Values are the mean±SD from three experiments. (*) p<0.05, (**) p<0.01. (K) Stimulating effect of H₂S on H₂O₂ degrading activity and SOD activity in crude extracts of wt and 3MSTdeficient E. coli cells. Total H₂O₂ degrading activity was measured as described in Shatalin et al., Proc. Natl. Acad. Sci. U.S.A. 105, 1009 (2008). Catalase activity at 100% is 30 mM H₂O₂ Values shown are the means±SEM from three experiments. SOD activity was measured using a tetrazolium-based assay kit. (L) The proposed mechanism of H₂S-mediated defense against antibiotics. Despite having different primary targets, many antibiotics kill bacteria by generating ROS (Asad and Leitão, J. Bacteriol. 173, 2562 (1991); Aruoma et al., J. Biol. Chem. 264, 20509 (1989)). The ability of H₂S-producing enzymes (CBS, CSE, and 3MST) to alleviate oxidative stress is achieved by several mechanisms: (i) depletion of free cysteine (Cys), which fuels the Fenton reaction; (ii) inhibition of the Fenton reaction by H₂S, which reacts with H₂O₂ and diminishes free Fe²⁺; (iii) stimulation of catalase and superoxide dismutase activities. Antibiotics stimulate the activities of CBS, CSE, and 3MST, thereby ensuring their specific defensive responses. (M) Effect of Cys and NaHS on Fenton-mediated DNA damage in vitro. As indicated, the supercoiled pBR322 plasmid (0.5 μg) was treated with 30 μM FeCl₃, 4 mM Cys, 200 μM NaHS, or 4 mM H₂O₂ in 20 mM Tris-HCl buffer (pH 8). After a 30-min incubation at room temperature, the reaction was stopped and separated in a 1% agarose gel. RF, relaxed form; SF, supercoiled form. (N) Dual protective effect of H₂S against oxidative stress: Catalase and SOD are required for prolonged defense against H₂O₂ toxicity mediated by NaHS but not for immediate protection. Wt, katE, and sodA E. coli cells were grown in Luria-Bertani broth (LB) to absorbance (optical density) OD₆₀₀ of ˜1.0, treated with NaHS (200 mM) for the indicated time intervals (min), followed by the addition of H₂O₂ (2 mM) for 10 min. Cell survival was determined by counting CFU and is shown as the mean TSD from three independent experiments.

FIG. 11 shows that H₂S-generating CBS/CSE becomes essential in the absence of bNOS. B. anthracis (34F2Δnos cbs/sce::Pspac) cells carrying the chromosomal copy of cbs/cse under the IPTG-inducible spac promoter were plated on LBA plates with or without 0.1 mM IPTG for overnight incubation at 37° C.

FIG. 12A-F show synergistic action of H₂₅ and NO in B. anthracis. (A) Compensatory induction of endogenous H₂S and NO. In vivo production of NO in response to deletion of CBS/CSE or cefuroxime (Cef) (20 mg/ml) challenge was detected using the Cu(II)-based NO fluorescent sensor (CuFL) (Lim et al., Nat. Chem. Biol. 2, 375 (2006)) (left bars). Cells were grown in LB to OD₆₀₀ of ˜0.5 followed by addition of freshly prepared CuFL (20 mM) and Cef. Fluorescence was measured in the total culture after 18 hours of incubation using a real-time fluorometer (PerkinElmer LS-55). H₂S was measured using Pb(Ac)₂ as in FIG. 4A (right bars). (B) H₂S induction in response to antibiotic (erythromycin) or H2O2 challenge. The plot shows β-galactosidase activity (Miller units) expressed by B. anthracis cells harboring a chromosomal transcriptional fusion of the cbs/cse promoter and leader region to a promoterless lacZ gene. Bacteria were grown in LB medium until OD₆₀₀ of ˜0.6 followed by the addition of 0.5 mg/ml erythromycin or 2 mM H₂O₂. The bottom panel shows PbS brown or black stain, which is proportional to the amount of H₂S produced. B. anthracis cells were grown in 96-well plates in LB+Cys (200 mM) covered with lead acetate-soaked paper by using a Bioscreen C automated growth analysis system. (C) H₂S induction in response to erythromycin or H₂O₂ challenge. E. coli cells were grown in 96 wells plates in LB+Cys (25 or 200 μM) covered with lead acetate soaked paper at 37° C. with aeration using a Bioscreen C automated growth analysis system. Em (2.5 μg/ml) or H₂O₂ (1 mM) were added at OD₆₀₀ ˜0.6 for 12 hours. PbS brown/black stain is proportional to the amount of H₂S. (D) Representative OD growth curves of wt (black curves), CBS/CSE-deficient (circles) or bNOS deficient (triangles) B. anthracis (Sterne) cells. Acriflavine, PAG/AOAA, NaSH, and the NOS inhibitor N-nitro-L-arginine methyl ester (L-NAME) were added as indicated. Cells were grown in triplicate at 37° C. with aeration using a Bioscreen C automated growth analysis system. The curves represent the averaged values (P<0.05). (E) Wild type E. coli cells protect 3MST-deficient cells from antibiotic toxicity. WT and ΔsseA cells were grown in LB+Cys (0.5 mM) to OD⁶⁰⁰ ˜0.6. Then aliquots containing equal amounts of cells were mixed together, washed, resuspended in M9 minimal media supplemented with Gm (5 μg/ml) and incubated for 30 min at 37° C. Cell survival was determined by counting CFU and is shown as the mean±SD from three independent experiments. (F) Protection of 3MST-deficient cells from Cys-mediated gentamicin toxicity by the thiol oxidizer diamide and NaHS. Cells grown in LB or LB+Cys (0.5 mM) to OD₆₀₀ ˜1.0 were washed in M9 minimal media supplemented with the thiol oxidizer diamide (25 μM) or NaHS (0.2 mM) or Cys (0.5 mM). After 3 min of incubation gentamicin (50 μg/ml) was added for 5 min. Cell survival was determined by counting CFU and is shown as the mean±SD from three independent experiments.

FIG. 13 shows that AOAA was able to potentiate, the effects of various antibiotics on cultures of Enterococcus faecalis. Bacterial cultures were prepared at initial inoculums of ˜5×10⁵ CFU/ml, and aliquots of 190 μl per well were dispensed. Ten μl of the indicated antibiotic solutions were added to each well. The starting concentration for each antibiotic solution was 2×MIC, with serial ½ dilutions. Two sets of dilutions were prepared, and 0.1 mM AOAA was added to one set. The plates were kept at 37° C. and 85% humidity for 20 hours.

### DETAILED DESCRIPTION OF THE INVENTION

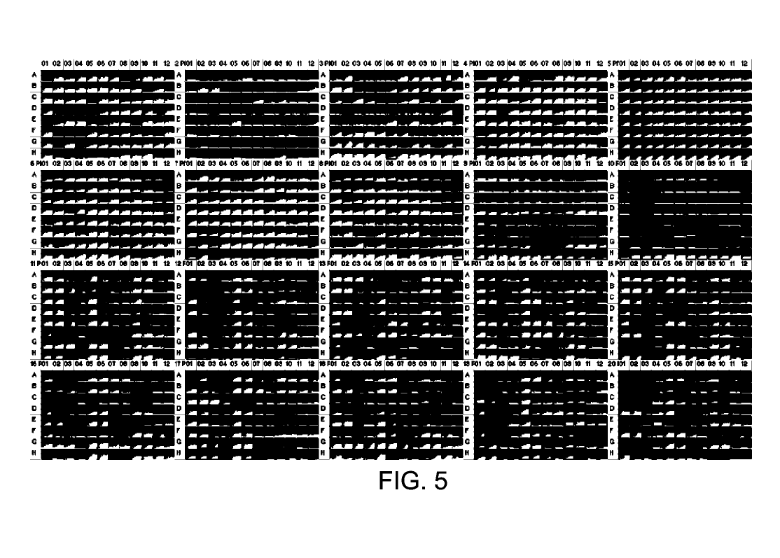

The present invention is based in part on the discovery that microbial H₂S is cytoprotective. As described in the Examples below, the present inventors have discovered that (1) bacterial cystathionine β-synthase (CBS), cystathionine γ-lyase (CSE) and 3-mercaptopyruvate sulfurtransferase (3MST) generate H₂S under normal growth conditions and protect bacteria against a broad range of antibiotics, and (2) that the mechanism of H₂S-mediated defense against antibiotics relies on protection against oxidative stress and the DNA-damaging Fenton reaction. As described herein, endogenous microbial H₂S can diminish the effectiveness of clinically used antibiotics. Thus, inhibition of this “gasoprotector” may be useful as an augmentation therapy against a broad range of microbial pathogens, including bacteria, fungi and protozoa. As microbial CBS, CSE, and 3MST diverge significantly from their mammalian counterparts (see, e.g., FIG. 5A-C), it may be possible to design specific inhibitors that target these enzymes but not their mammalian orthologs.

In one aspect, the invention provides a method for treating a subject having a microbial infection, said method comprising administering to said subject a therapeutically effective amount of at least one inhibitor of endogenous H₂S production by an organism (e.g., pathogenic bacteria, fungi, protozoa) causing the microbial infection. In one embodiment, the method further comprises administering a second antimicrobial compound.

In another aspect, the invention provides a method for enhancing efficacy of an antimicrobial treatment in a subject having a microbial infection, wherein said antimicrobial treatment comprises administering to the subject a first antimicrobial compound that becomes compromised by H₂S or natural products of H₂S metabolism in vivo, said method comprising co-administering said first compound with a therapeutically effective amount of a second compound which second compound is an inhibitor of endogenous H₂S production by an organism (e.g., pathogenic bacteria, fungi, protozoa) causing the microbial infection.

In a separate aspect, the invention provides a method for sensitizing a microbial pathogen (e.g., pathogenic bacteria, fungi, protozoa) to oxidative damage comprising administering to said pathogen an effective amount of at least one inhibitor of endogenous H₂S production by said pathogen. In one embodiment, the pathogen is in a subject and the inhibitor of endogenous H₂S production is administered to the subject. Such sensitization can be important, for example, for facilitating mammal's own defense against infections. When mammalian immune system attempts to kill pathogens via oxidative stress, pathogens′ H₂S provides protection against the immune response.

In one embodiment, the inhibitor of endogenous H₂S production useful in the methods of the present invention inhibits an H₂S-generating enzyme within the organism causing the microbial infection. For example, AOAA and hydroxylamine can be used to inhibit CBS; PAG and β-cyano-L-alanine can be used to inhibit CSE; and aspartate and derivatives thereof can be used to inhibit 3MST.

In one embodiment, the inhibitor of endogenous H₂S production inhibits microbial cystathionine β-synthase (CBS) or paralogs thereof. In a specific embodiment, such CBS inhibitor can be co-administered (simultaneously in the same or separate compositions or sequentially) with an inhibitor of microbial cystathionine γ-lyase (CSE) or paralogs thereof. In a separate embodiment, the inhibitor of endogenous H₂S production inhibits microbial 3-mercaptopyruvate sulfurtransferase (3MST) or paralogs thereof. In another embodiment, the inhibitor of endogenous H₂S production inhibits microbial cystathionine γ-lyase (CSE) or paralogs thereof.

In one embodiment, the inhibitor of endogenous H₂S production is amino-oxyacetate (AOAA). In another embodiment, the inhibitor of endogenous H₂S production is hydroxylamine. In yet another embodiment, the inhibitor of endogenous H₂S production is D,L-propargylglycine (PAG). In a further embodiment, the inhibitor of endogenous H₂S production is β-cyano-L-alanine. In another embodiment, the inhibitor of endogenous H₂S production is aspartate or a derivative thereof. In an additional embodiment, the inhibitor of endogenous H₂S production is homocysteine. In one embodiment, the inhibitors of endogenous H₂S production useful in the methods of the present invention selectively inhibit H₂S-generating enzyme(s) within the organism causing the microbial infection, but not H₂S-generating enzyme(s) in the cells of the subject being treated. Such selective inhibitors can be identified using various screening methods known in the art, e.g., as described in International Application Publication No. WO 2011/130181.

The methods of the present invention are useful for treating acute or chronic infections caused by various microorganisms, including, e.g., bacteria, fungi and protozoa. Non-limiting examples of encompassed bacterial genera include, e.g., Bacillus, Brucella, Clostridium, Enterococcus, Escherichia, Francisella, Helicobacter, Klebsiella, Legionella, Listeria, Mycobacterium, Pseudomonas, Salmonella, Shigella, Staphylococcus, Streptococcus, Trypanasoma, Vibrio, and Yersinia. In one specific embodiment, bacteria are from a species selected from the group consisting of E. coli, S. aureus, B. anthracis, and P. aeruginosa.

Non-limiting examples of microbial infectious diseases which can be treated by the methods of the present invention include, e.g., pneumonia, bronchitis, diphtheria, pertussis (whooping cough), tetanus, endocarditis, sepsis, bacterial gastroenteritis, cholera, tuberculosis, gonorrhea, chlamydia, syphilis, bacterial meningitis, malaria, trachoma, leishmaniasis, chagas disease, trichomoniasis, lyme disease, and leprosy.

The methods of the present invention can be used to treat infections in various animals, including, e.g., mammals, birds and fish. In one specific embodiment, the methods of the present invention are used to treat infections in humans.

Examples of antimicrobial compounds that can be used in the methods provided herein include, without limitation, quinolones, acridines, phenothiazines, aminoglycosides, macrolides, amphenicols, steroids, ansamycins, antifolates, polymyxins, glycopeptides, cephalosporins, and lactams. For example, a method as provided herein can include administration of an antimicrobial compounds selected from the group consisting of novobiocin, norfloxacin, nalidixic acid, oxolinic acid, acriflavine, 9-aminoacridine, proflavine, trifluoperazine, promethazine, chlorpromazine, streptomycin, apramycin, tylosin, oleandomycin, erythromycin, josamycin, spirathycin, troleandomycin, chloramphenicol, fusidic acid, rifamycin SV, trimethoprim, 2,4-diamino-6,7-diisopropylpteridine, polymyxin B, colistin, vancomycin, cefsulodin, cephalothin, cefoperazone, oxacillin, nafcillin, phenethicillin, penicillin G, cloxacillin, moxalactam, and carbenicillin. Other antimicrobial compounds that can be used include, for example, those listed in Tables 1 and 2, below.

In some embodiments, the methods provided herein also can include further administering an inhibitor of endogenous microbial NO production or an NO scavenger.

Non-limiting examples of useful inhibitors of endogenous NO production include L-arginine, NG-monomethyl-L-arginine (NMMA), NG-nitro-L-arginine methyl ester (NAME), NG-nitro-L-arginine (NNA), NG-amino-L-arginine (NAA), NG,NG-dimethylarginine (asymmetric dimethylarginine, called ADMA), L-Thiocitrulline, S-methyl-L-Thiocitrulline, diphenyleneiodonium chloride, 2-(4-carboxyphenyl)-4,4,5,5-tetramethylimidazoline-1-oxy 3-oxide, 7-nitroindazole, N(5)-(1-iminoethyl)-L-ornithine, aminoguanidine, canavanine, ebselen, S-methyl-L-citrulline, S-methylisourea, and 2-mercaptoethylguanidine. See, also, the inhibitors disclosed in Hobbs et al. (1999) Annu. Rev. Pharmacol. Toxicol. 39:191-220; and Salard et al. (2006) J. Inorg. Biochem. 100:2024-2033. In some embodiments, iNOS-specific inhibitors can be particularly useful.

NO scavengers include, without limitation, non-heme iron-containing peptides or proteins, porphyrins, metalloporphyrins, dithiocarbamates, dimercaptosuccinic acid, phenanthroline, desferrioxamine, pyridoxal isonicotinoyl hydrazone (PIH), 1,2-dimethyl-3hydroxypyrid-4-one (L1), [+] 1,2-bis(3,5-dioxopiperazine-lyl)propane (ICRF-187), 2-(4-carboxyphenyl)-4,5-dihydro-4,4,5,5-tetramethyl-1H-imidazolyl-1-oxy-3-oxide (Carboxy-PTIO), and the like. A particularly useful NO scavenger may be a perfluorocarbon emulsion, as disclosed in Rafikova et al. (2004) Circulation 110(23):3573-3580.

In conjunction with the methods of the present invention, provided herein are various combination pharmaceutical compositions. In one embodiment, the invention provides a pharmaceutical composition comprising (i) an antimicrobial compound that becomes compromised by H₂S or natural products of H₂S metabolism in vivo and (ii) an inhibitor of microbial endogenous H₂S production. In a specific embodiment, this composition further comprises (iii) an inhibitor of microbial endogenous nitric oxide (NO) production or a NO scavenger. In a separate embodiment, the invention provides a pharmaceutical composition comprising (i) an inhibitor of microbial endogenous H₂S production and (ii) an inhibitor of microbial endogenous nitric oxide (NO) production or a NO scavenger.

The compounds described herein can be formulated into pharmaceutical compositions and formulations. Compounds therefore can be admixed, encapsulated, conjugated or otherwise associated with one or more pharmaceutically acceptable carriers and/or other molecules, molecular structures, or mixtures of compounds such as, for example, liposomes, polyethylene glycol, receptor targeted molecules, for oral, topical or other formulations, for assisting in uptake, distribution and/or absorption.

Methods for formulating and subsequently administering therapeutic compositions are well known to those skilled in the art. Dosing generally is dependent on the severity and responsiveness of the disease state to be treated, with the course of treatment lasting from several days to several months, or until a cure is effected or a diminution of the disease state is achieved. Persons of ordinary skill in the art routinely determine optimum dosages, dosing methodologies and repetition rates. Optimum dosages can vary depending on the relative potency of individual compounds, and can generally be estimated based on EC₅₀ found to be effective in in vitro and in vivo animal models. Typically, dosage is from 0.01 μg to 100 g per kg of body weight, and may be given once or more daily, biweekly, weekly, monthly, or even less often. Following successful treatment, it may be desirable to have the patient undergo maintenance therapy to prevent the recurrence of the disease state.

Pharmaceutical compositions can be administered by a number of methods, depending upon whether local or systemic treatment is desired and upon the area to be treated. Administration can be, for example, topical (e.g., transdermal, sublingual, ophthalmic, or intranasal); pulmonary (e.g., by inhalation or insufflation of powders or aerosols); oral; or parenteral (e.g., by subcutaneous, intrathecal, intraventricular, intramuscular, or intraperitoneal injection, or by intravenous drip). Administration can be rapid (e.g., by injection) or can occur over a period of time (e.g., by slow infusion or administration of slow release formulations). For treating tissues in the central nervous system, compounds can be administered by injection or infusion into the cerebrospinal fluid, typically with one or more agents capable of promoting penetration of the polypeptides across the blood-brain barrier.

### DEFINITIONS

The term, “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

As used herein, the term “paralogs” (of CBS, CSE, and 3MST) refers to homologous sequences within a microbial genome which result from a gene duplication event. The CBS, CSE, and 3MST paralogs encompassed by the present invention may have altered functional properties as compared to CBS, CSE, and 3MST, but are still involved in or affect the endogenous microbial H₂S production.

As used herein, the term “compromised by H₂S or natural products of H₂S metabolism” refers to antimicrobial compounds which become less effective in the presence of H₂S or natural products of H₂S metabolism (i.e., any sulfur-containing organic [e.g. cysteine, glutathione] or inorganic [e.g., FeS, NaHS] products of H₂S metabolism). This term encompasses complete and partial loss of antimicrobial activity of a given compound.

In the context of the present invention insofar as it relates to any of the disease conditions recited herein (e.g., infection), the terms “treat,” “treatment,” and the like mean to relieve or alleviate at least one symptom associated with such condition, or to slow or reverse the progression of such condition.

The terms “administering” or “administration” are intended to encompass all means for directly and indirectly delivering a compound to its intended site of action. The compounds of the present invention can be administered locally to the affected site (e.g., by direct injection into the affected tissue) or systemically. The term “systemic” as used herein includes parenteral, topical, oral, spray inhalation, rectal, nasal, and buccal administration. Parenteral administration includes subcutaneous, intravenous, intramuscular, intra-articular, intra-synovial, intrasternal, intrathecal, intrahepatic, intralesional, and intracranial administration.

As used herein, the term “therapeutically effective” applied to dose or amount refers to that quantity of a compound or pharmaceutical composition that is sufficient to result in a desired activity upon administration to a subject in need thereof. Within the context of the present invention, the term “therapeutically effective” refers to that quantity of a compound (e.g., an inhibitor of endogenous H₂S production or its combination with a second antimicrobial compound and/or an inhibitor of endogenous microbial NO production or an NO scavenger) or pharmaceutical composition containing such compound that is sufficient to delay manifestation, arrest the progression, relieve or alleviate at least one aspect of an infection. Note that when a combination of active ingredients is administered the effective amount of the combination may or may not include amounts of each ingredient that would have been effective if administered individually.

The phrase “pharmaceutically acceptable,” as used in connection with compositions of the invention, refers to molecular entities and other ingredients of such compositions that are physiologically tolerable and do not typically produce untoward reactions when administered to an animal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The term “subject” refers to any animal, including, e.g., mammals, birds and fish, and, in particular, humans.

As used in this specification and the appended claims, the singular forms “a,” “an,” and “the” include plural references unless the context clearly dictates otherwise.

In accordance with the present invention, there may be employed conventional molecular biology, microbiology, and recombinant DNA techniques within the skill of the art. See, e.g., Sambrook, Fritsch and Maniatis, Molecular Cloning: A Laboratory Manual, 2nd ed., Cold Spring Harbor Laboratory Press, Cold Spring Harbor, N.Y., 1989; DNA Cloning: A Practical Approach, Volumes I and II (Glover ed. 1985); Oligonucleotide Synthesis (Gait ed. 1984); Nucleic Acid Hybridization (Hames and Higgins eds. 1985); Transcription and Translation (Hames and Higgins eds. 1984); Animal Cell Culture (Freshney ed. 1986); Immobilized Cells and Enzymes (IRL Press, 1986); B. Perbal, A Practical Guide To Molecular Cloning (1984); and Ausubel et al. eds., Current Protocols in Molecular Biology, John Wiley and Sons, Inc. 1994, among others.

The invention will be further described in the following examples, which do not limit the scope of the invention described in the claims.

### EXAMPLES

### Example 1

### Materials and Methods

Strains and Growth Conditions:

E. coli, S. aureus, and P. aeruginosa strains were grown in Luria-Bertani (LB) broth or on LB plates supplemented with 1.5% Bacto agar at 37° C. B. anthracis strains were grown in BHI media supplemented with glycerol at 37° C. Construction of sseA deletion and sseA overexpression strains of E. coli were produced according to Datsenko and Wanner (34). Briefly, an attL-Km^{R}-attR cassette (35) was amplified with primers 5′-atgcgtgagaatttacgttatgtaattcagtatcaccgctcaagttagtataaaaaagct-3′ (SEQ ID NO: 1) and 5′-ttatttcactggctcaaccggtaaatctgcccgcgtgaagcctgcttttttatactaag-3′ (SEQ ID NO: 2) and transformed into an MG1655 strain containing pKD46. sseA mutants were selected on LB agar supplemented with Km and verified by PCR with primers 5′-attagcatgcaggcggctac-3′ (SEQ ID NO: 3) and 5′-tgctggaaaggccgcgaa-3′ (SEQ ID NO: 4).

To construct an sseA overexpression strain, the sseA promoter was substituted for P_{Ltet-O1} (24). Briefly, an attL-Cm^{R}-attR cassette (35) was amplified with primers 5′-cgcagatcttgaagcctgcttttttatac-3′ (SEQ ID NO: 5) and 5′-cgctcaagttagtataaaaaagct-3′ (SEQ ID NO: 6) and ligated with P_{Ltet-O}1 obtained with primers 5′-cgcagatctcgagtccctatcagtg-3′ (SEQ ID NO: 7) and 5′-aatttctcctctttccatgg-3′ (SEQ ID NO: 8). A P_{Ltet-O1-} attL-Cm^{R}-attR cassette was then amplified with primers 5′-ccctgccacaatggcccgttagcaacgtcgaataacgctcaagttagtataaaaaagct-3′ (SEQ ID NO: 9) and 5′-gtgatactgaattacataacgtaaattctcacgcatggtacctttctcctattaatga-3′ (SEQ ID NO: 10). The first primer contains the upstream region of sseA and sequence of attR, while the second primer contains the coding region of sseA and sequence of P_{Ltet-O}1. The PCR fragment was transformed into MG1655 containing pKD46. CmR clones were tested in the presence of the P_{Ltet-O1}-attL-Cm^{R}-ttR cassette by PCR with primers 5′-attagcatgcaggcggctac-3′ (SEQ ID NO: 11) and 5′-gcaaaagaggctgatttggct-3′ (SEQ ID NO: 12).

E. coli metB, metC, malY, cysM, cysK, and tnaA deletion mutants were obtained from the E. coli Keio Knockout Collection (Thermo Scientific).

The B. anthracis nos deletion strain Sterne 34F2 was described previously (28). The conditional KO (CO) of cbs/cse in B. anthracis 34F2 was made using a protocol described in Fisher and Hanna, J. Bacteriol. 187, 8055 (2005). The 0.5 kb fragment of cbs from −20 to 480 with respect to the translational start site was amplified from 34F2 genomic DNA by using primers designed for use in the Gateway cloning system (Invitrogen). The upstream primer had the sequence 5′-ggggacaagtttgtacaaaaaagcaggctAAGGGGGAGAACACGATGAATG-3′ (SEQ ID NO: 19; RBS and start codon underlined), and the downstream primer had the sequence 5′-ggggaccactttgtacaagaaagctgggtgtgccgaccaaagttcaggac-3′ (SEQ ID NO: 20). The resulting amplicon was transferred to pDONRtet using standard BP reaction conditions (Invitrogen), followed by transformation of competent DH10B E. coli and selection on LB plates containing 10 μg/ml of tetracycline. Cloned amplicons were transferred to pNFd13 (Ts pE194 ori) under the spac promoter using the standard LR reaction (Invitrogen), followed by transformation of DH10B E. coli and selection on LB plates containing 50 μg/ml Km. Integration of the plasmid into the targeted B. anthracis locus was done at 39° C. essentially as described in Fisher and Hanna, J. Bacteriol. 187, 8055 (2005). The nos deletion in the resulting 34F2 cbs/cse::pNFd13 strain was obtained as described in Shatalin et al., Proc. Natl. Acad. Sci. U.S.A. 105, 1009 (2008), except that a spectinomycin cassette was used instead of Km.

The P. aeruginosa PA-14 wt and PA-14 cbs mutant strains were obtained from Massachusetts General Hospital's P. aeruginosa mutants collection (Department of Molecular Biology, Richard B. Simches Research Center.

Generation of Growth Curves:

Growth curves were obtained on a Bioscreen C automated growth analysis system (Oy Growth Curves Ab Ltd.). Subcultures of specified strains Were grown overnight at 30° C., diluted 1:100 in fresh media and grown to OD₆₀₀ ˜0.8 at 37° C. Cultures were then diluted 1:100 in LB or BHI media with appropriate antibiotics or reagents as described in the text or figure legends. 300 μl of each mixture was inoculated into honeycomb wells in triplicate and grown at 37° C. with maximum shaking on the platform of the Bioscreen C instrument. OD₆₀₀ values determined were recorded automatically at different times and the means of the triplicate cultures were plotted.

Generation of Survival Curves:

Overnight cultures were inoculated into LB supplemented with 0.5 mM Cys and grown at 37° C. to ˜2×10⁷ cells per ml. Then cells were diluted 100 times in sterile 0.9% NaCl and treated by Gm (50 μg/ml) or Km (250 μg/ml) or Cm (70 μg/ml for P. aeruginosa strains and 35 μg/ml for other strains) at room temperature in, the presence or absence of 0.2 mM of NaHS. CBS/CSE inhibitors, 0.5 mM PAG and 50 μM AOAA, were used in experiments with WT S. aureus and P. aeruginosa. At different times samples of cells were placed on LB agar and incubated at 37° C. for 16-18 hours. Cell survival was determined by counting CFU and is shown as the mean±SD from three independent experiments.

H₂S Detection:

To monitor H₂S production in wild type (wt) and mutant cells, a lead acetate detection method was used (7). Paper strips saturated with 2% Pb(Ac)₂ were affixed to the inner wall of a culture tube, above the level of a liquid culture of wild type or mutant bacteria. Overnight cultures were diluted 1:50 in LB or BHI without or with Cys (25-500 μM), and incubated 18-20 hours at 37° C. with aeration. Stained paper strips were scanned and quantified with an AlphaImager (Imgen Technologies; Alexandria, Va.). The results were normalized per ODs.

Assay of Chromosomal DNA Damage:

The assay was done as described previously (28). Cells were grown to OD₆₀₀=1.0 in LB media and treated with H₂O₂ (1 mM—E. coli; 2 mM—B. anthracis; 2.5 mM—P. aeruginosa) or antibiotics Em (1 Mg/ml—B. anthracis); Ap (10 μg/ml—E. coli; 125 μg/ml—P. aeruginosa) for 4 hours. Cells were equalized to OD₆₀₀=1.0 by addition of fresh media and the cells from 2 ml of the equalized cultures were harvested by centrifugation. Total genomic DNA was isolated from the bacteria pellets with a QIAamp DNA Mini Kit (Qiagen; Valencia, Calif.) or according the GenElute Bacterial Genomic DNA Kit protocol for Gram Positive Bacteria (Sigma; St. Louis, Mo.). DNA was extracted with phenol/chloroform and quantified with the PicoGreen dsDNA quantitation reagent (Molecular Probes®; Invitrogen; Carlsbad, Calif.) and λ phage DNA as a standard. An arbitrarily chosen, 10-kb, 6-kb, and 5.8-kb fragments of E. coli, B. anthracis and P. aeruginose genomes, respectively, were used for qPCR. Primer sequences were as follows: E. coli 5′-ttccattgggatgtagatgctg-3′ (forward; SEQ ID NO: 13) and 5′-ggtaaaagagtcaagggaagaacc-3′ (reverse; SEQ ID NO: 14), B. anthracis 5′-acgattgacttctctcacttcggt-3′ (forward; SEQ ID NO: 15) and 5′-aaacatttgctcttgatgtcctgga-3′ (reverse; SEQ ID NO: 16), P. aeruginose 5′-ctttgcaacctgtacatgccttg-3′ (forward; SEQ ID NO: 17) and 5′-catcgtagtagttgatcggatggac-3′ (reverse; SEQ ID NO: 18). PCR was performed with Phusion DNA polymerase (Finnenzymes). The 50-μl PCR mixture contained 0.5 ng of genomic DNA as a template, 1.5 μM primers, 200 μM dNTPs (Fermentas), Phusion GC PCR buffer, and 0.5 μl of DNA polymerase. DNA was subjected to 29 to Cycles of PCR (98° C. for 30 seconds, 53° C. for 30 seconds, and 72° C. for 3 or 9 minutes). PCR products were separated by electrophoresis in a 0.8% or 1% agarose gel, stained with ethidium bromide, scanned, and quantified with an AlphaImager (Imgen Technologies).

Electrophoresis Assay of DNA Damage In Vitro (Fenton Reaction):

The nicking reaction mixture contained 0.1 μg pBR322 plasmid DNA in 20 mM Tris HCl buffer, pH 8.0, 30 μM FeCl₃, 4 mM Cys and 4 mM H₂O₂, in the presence or absence of 0.2 mM NaHS. After incubating the mixture at room temperature for 15 min the DNA samples were applied to an 0.8% agarose gel in a TAE buffer system, and electrophoresis was performed at 80 V for 30 min. Following electrophoresis, gels were stained with ethidium bromide for 30 min. After washing, the bands were visualized in a UV transilluminator. The modification of the fluorescence intensity of the bands is due to DNA strand breakage that leads to a decrease in the proportion of the supercoiled form and to an increase in the relaxed form produced by a nick in one strand. Pulse Field Gel Electrophoresis (PFGE): E. coli cells were grown to OD₆₀₀-0.8 at 32° C. 200 μM of NaHS, 10 μg/ml of Ap, or 1 mM of peroxide were added as indicated and cells were allowed to grow at 37° C. for 4 more hours. Agarose plugs were prepared using 3×10⁸ cells/plug and treated with lysozyme and Proteinase K according to the BioRad CHEF protocol (Bio-Rad Laboratories; Hercules, Calif.). Linearized 4.6 Mb E. coli chromosomal DNA marker was made by digesting the genomic DNA plug of SMR8476 strain with I-SceI endonuclease. DNA fragments were separated on a 1% agarose gel in 0.5×TBE at 14° C. for 24 hours at 6 V/cm using a Bio-Rad CHEF-DR II angle system with a 2.8-26.3 second linear switch time ramp. Gels were stained with EtBr and visualized with UV-trans-illumination. DNA was quantified (integrated density of the linear products) using Image) software.

CBS/CSE Expression Assay:

B. anthracic Stern 34F2 cbs/cse::pNFd13 cells (see above) carrying the lacZ reporter under the native cbs promoter were used. To monitor expression of cbs-lacZ fusions, bacteria were grown overnight at 37° C. in LB medium, washed and diluted 1:25 in fresh medium containing erythromycin or H₂O₂ at the specified concentrations. Cultures were incubated for 2-2.5 hr at 37° C. (OD₆₀₀=0.5-0.6) before measurement of β-galactosidase activity. Shown β-galactosidase activities correspond to mean values from at least three independent experiments. Error bars correspond to the standard deviation.

Assays of the SOS Response:

Overnight cultures of strain 10973 harboring a recA′-gfp chromosomal fusion (18) was grown in Luria broth with kanamycin (20 μg/ml). The cultures were diluted in LB to OD₆₀₀=0.1 and grown at 37° C. to an OD₆₀₀ ˜1.0. Asp (3 mM), Gm (5 μg/ml) and NaHS (200 μM) were added at OD₆₀₀=0.5. Fluorescence was measured (Ex. wave length: 480 nm and Em. wavelength: 520 nm) in a Perkin Elmer LS55 spectrofluorometer (Perkin Elmer; Waltham, Mass.). Fluorescence values were normalized to OD₆₀₀ values and plotted.

Catalase Activity Assay:

Degradation of H₂O₂ was monitored in real time as a decrease in absorbance at 240 nm (37). Aliquots of extracts to be monitored or of pure catalase were mixed with 50 mM phosphate buffer (pH 7.0) and placed into a 1 ml quartz cuvette. 40 mM H₂O₂ solution was added and the kinetics of its degradation recorded. Total H₂O₂ degrading activity was measured as the decrease of H₂O₂ concentration per milligram of total protein per second. OD₂₄₀ was converted to the concentration of H₂O₂ according to the calibration curve (10 mM H₂O₂=0.36 OD₂₄₀).

Superoxide Dismutase (SOD) Activity Assay:

Superoxide dismutase activity of cell extracts was determined using the Cayman Chemical Company Superoxide Dismutase Assay Kit (Cat#706002; Cayman Chemical Co.; Ann Arbor, Mich.) according to the Manufacture's recommendations.

Chemicals and Reagents:

All chemicals were from Sigma, except PYO and the Superoxide dismutase assay kit, which were purchased from Cayman. The CuFL NO sensor was provided by Dr. S. Lippard, MIT. See, Shatalin et al., Proc. Natl. Acad. Sci. U.S.A. 105, 1009 (2008).

### Example 2

### Bacterial CBS CSE and 3MST Generate H₂S Under Normal Growth Conditions

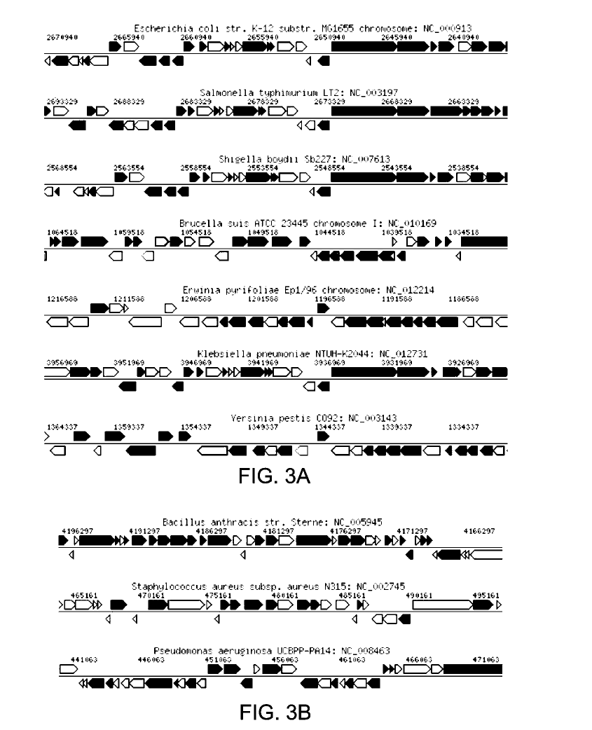

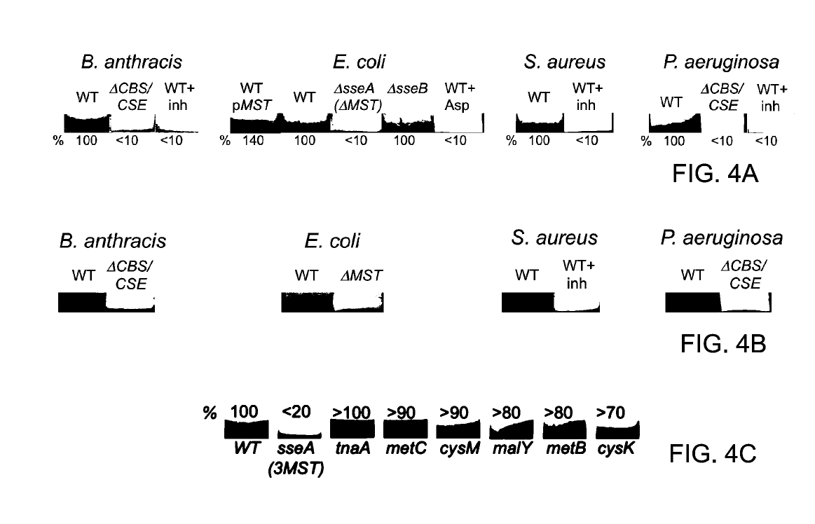

To determine whether CBS, CSE, or 3MST produces H₂S in bacteria, each enzyme was inactivated genetically or chemically in four clinically relevant and evolutionarily distant pathogenic species: Bacillus anthracis (Sterne), Pseudomonas aeruginosa (PA14), Staphylococcus aureus (MSSA RN4220 and MRSA MW2), and Escherichia coli (MG1655). The first three species have the CBS/CSE operon, but not 3MST, whereas E. coli carries 3MST, but not CBS/CSE. The chromosomal organization of H₂S genes (FIG. 3) and the strategy used for their replacement prevented any polar effects. H₂S production was monitored in wild-type (wt) and mutant cells using lead acetate [Pb(Ac)₂], which reacts specifically with H2S to form a brown lead sulfide stain. The rate of change of staining on a Pb(Ac)₂-soaked paper strip is directly proportional to the concentration of H₂S (Forbes, Bailey and Scott's Diagnostic Microbiology (Mosby, St. Louis, Mo., ed. 10, 1998)). Deletion of CBS/CSE in B. anthracis and P. aerugenosa or 3MST in E. coli greatly decreased or eliminated PbS staining (FIG. 4A). Similar results were obtained when DL-propargylglycine (PAG), aminooxyacetate (AOAA), or Asp were used, respectively, as specific inhibitors of CSE, CBS, or 3MST (FIG. 4A). Addition of Cys markedly increased PbS staining for all wt, but not CSE-CBS- or 3MST-deficient bacteria (FIG. 4B). In addition, overexpression of the chromosomal 3MST gene from a strong pLtetO-1 promoter in E. coli resulted in increased production of H₂S (FIG. 4A). Based on the above data, it can be concluded that all three enzymes produce H₂S endogenously from Cys during exponential growth of bacteria in rich media.

Further experiments demonstrated that 3MST is the major source of H₂S in E. coli. In addition to 3MST (sseA), the E. coli genome encodes several other enzymes that could potentially generate H₂S. These include cystathionine-γ-synthase MetB, cystathionine-β-synthases MetC and MalY, and CysM, CysK, and TnaA, which all are cysteine desulfurases (27). Pb(Ac)₂ analysis of individual strains harboring knockouts of each of these genes indicates that 3MST is the major source of H₂S production under specified growth conditions (FIG. 4C).

### Example 3

### Endogenous H₂S Protects Bacteria Against a Broad Range of Antibiotics

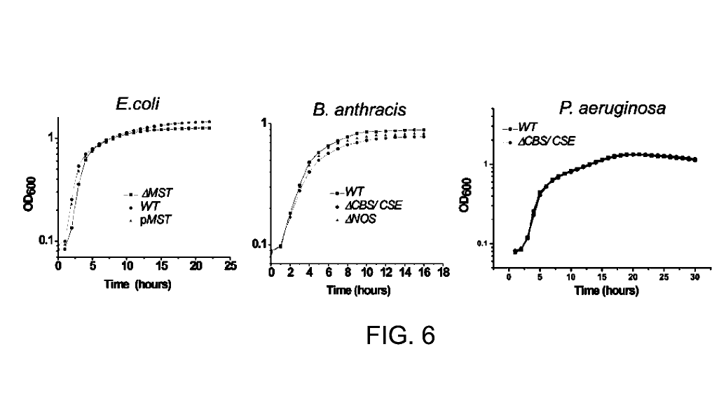

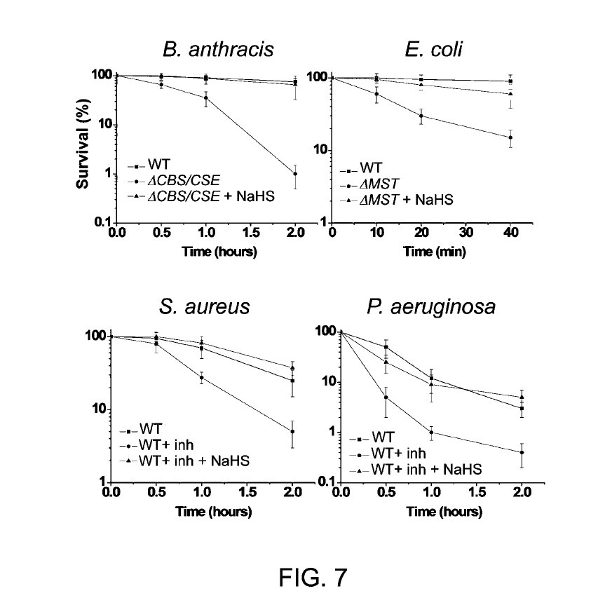

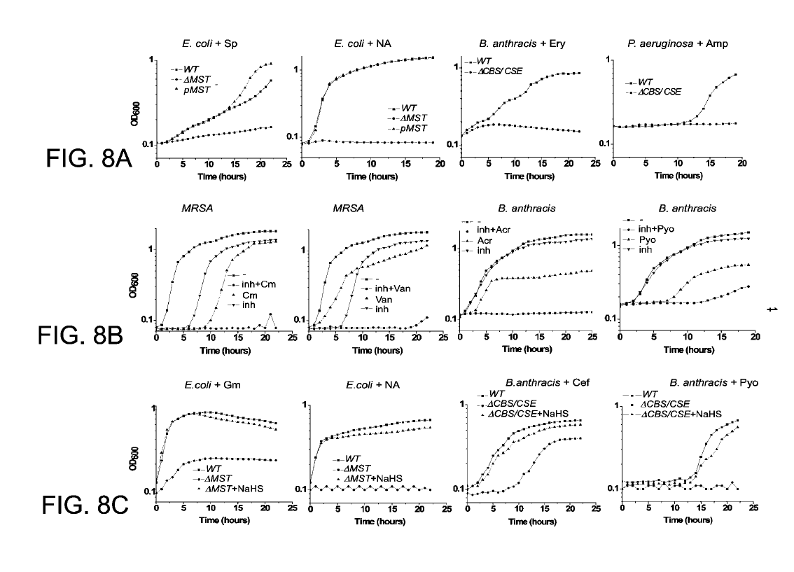

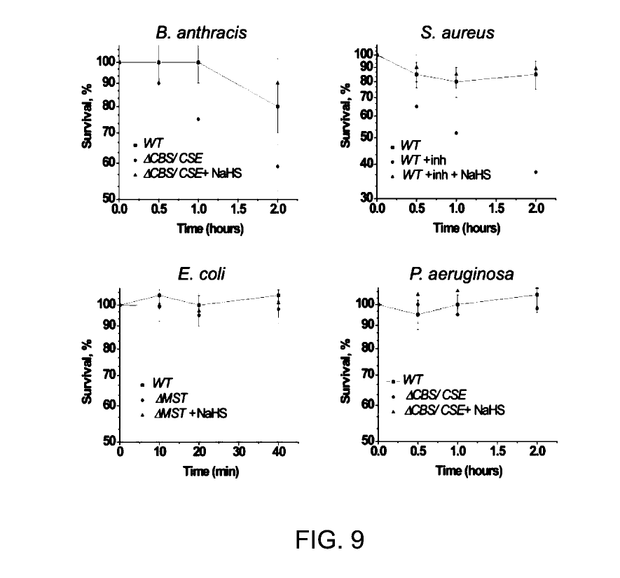

To elucidate the physiological role of H₂S, wt and 3MST-deficient E. coli were compared in a phenotype microarray (PMA) (FIG. 5 and Table 1). Whereas these strains showed little or no growth defects (FIG. 6), a large number of antibiotics, highly diverse in structure and function, preferentially suppressed growth of 3MST-deficient cells (Tables 1 and 2). The killing and growth curves obtained for wt and 3MST and CBS/CSE mutant E. coli, P. aerugenosa, S. aureus, and B. anthracis in the presence of several representative antibiotics confirmed the results of the screen and generalized them to both Gram-positive and -negative species (FIGS. 7-9). 3MST overexpression resulted in increased resistance to spectinomycin (FIG. 8A), whereas chemical inhibition of CBS, CSE, or 3MST rendered bacteria more sensitive to different antibiotics (FIGS. 7 and 8B). An H₂S donor, NaHS, suppressed the antibiotic sensitivity of CBS-CSE- and 3MST-deficient cells (FIGS. 7, 8C and 9). Taken together, these results establish that endogenously produced H₂S confers antibiotic resistance.

### Example 4

### H₂S Protects Bacteria Against Oxidative Stress and Antibiotic-Inflicted Oxidative Damage

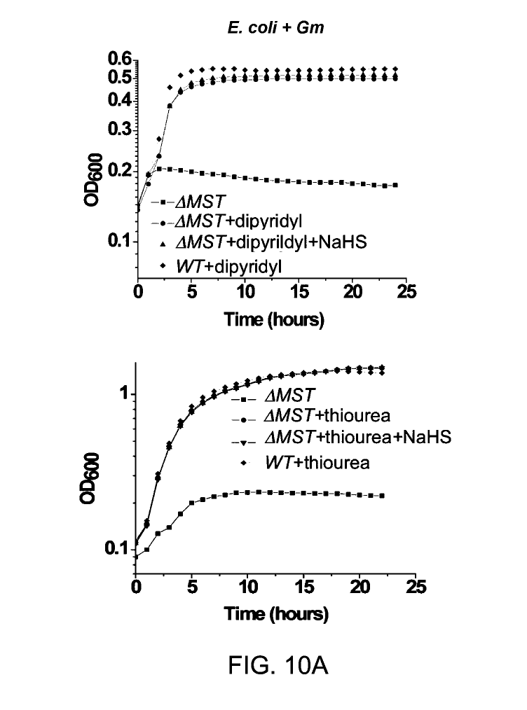

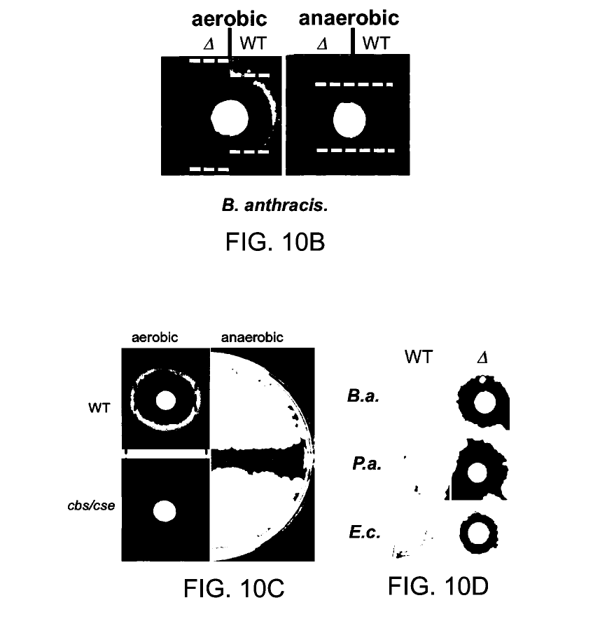

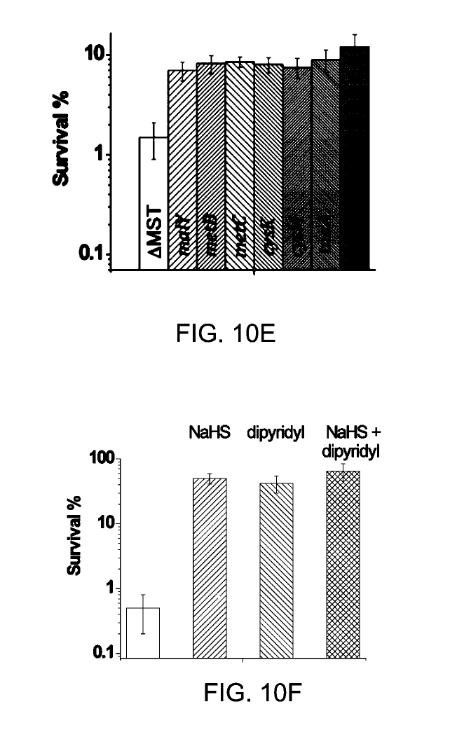

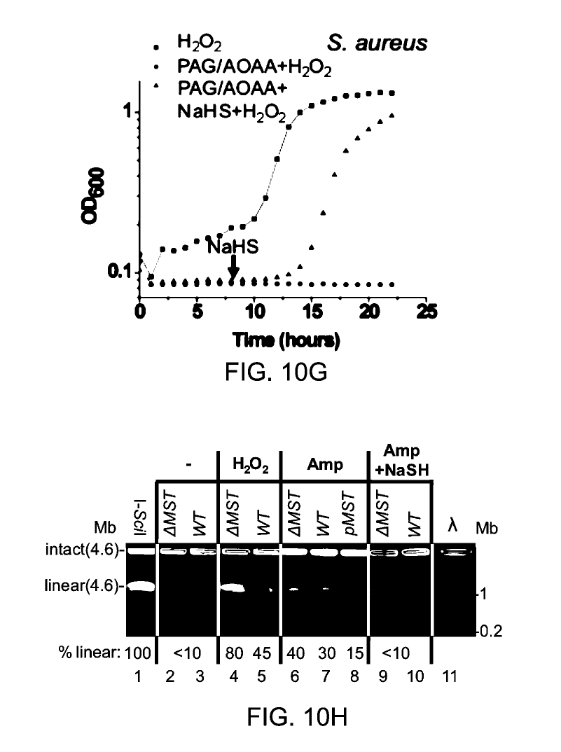

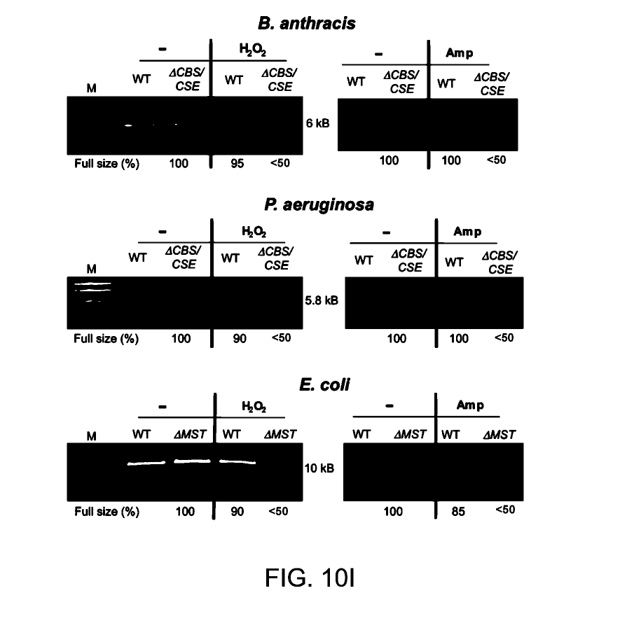

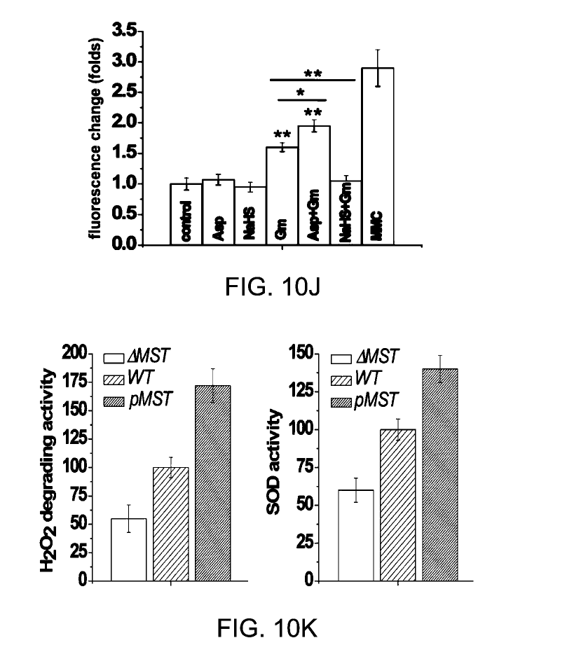

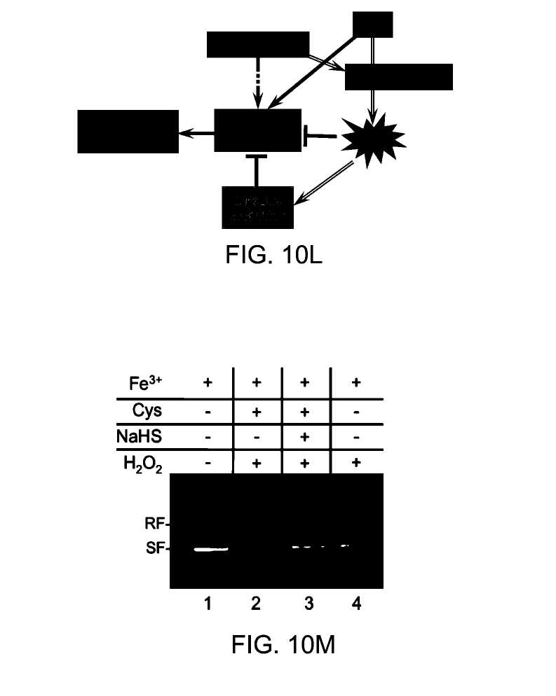

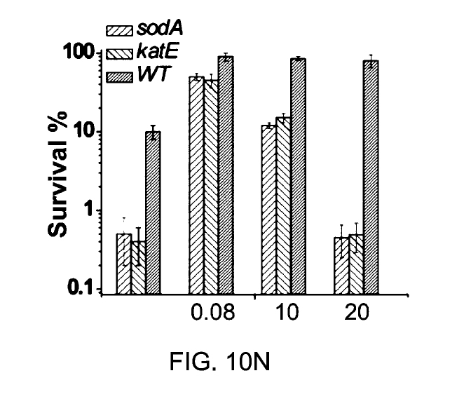

H₂S-mediated cytoprotection resembles that of NO, which defends certain Gram-positive bacteria against some of the same antibiotics as does H₂S (I. Gusarov, K. Shatalin, M. Starodubtseva, E. Nudler, Science 325, 1380 (2009)). NO-mediated protection relies, in part, on its ability to defend bacteria against oxidative stress imposed by antibiotics (I. Gusarov, E. Nudler, Proc. Natl. Acad. Sci. U.S.A. 102, 13855 (2005); K. Shatalin et al., Proc. Natl. Acad. Sci. U.S.A. 105, 1009 (2008); and 1. Gusarov, K. Shatalin, M. Starodubtseva, E. Nudler, Science 325, 1380 (2009)). To examine whether H₂S acts by a similar mechanism, detailed analyses of its effect on bacterial killing by representative antibiotics (gentamicin (Gm), ampicillin (Ap), and nalidixic acid (NA)) were conducted (FIG. 10). All three antibiotics have been shown to exert their bactericidal effect via oxidative stress (I. Gusarov, K. Shatalin, M. Starodubtseva, E. Nudler, Science 325, 1380 (2009); and M. A. Kohanski, D. J. Dwyer, B. Hayete, C. A. Lawrence, J. J. Collins, Cell 130, 797 (2007)). Indeed, pretreatment of cells with 2,2′-dipyridyl, an iron chelator that suppresses the damaging Fenton reaction (N. R. Asad, A. C. Leitão, J. Bacteriol. 173, 2562 (1991)), or the hydroxyl radical scavenger thiourea, substantially decreased the toxicity of Gm (FIG. 10A). Wild type and H₂S-deficient cells became equally resistant to Gm in the presence of dipyridyl or thiourea (FIG. 10A). Moreover, the H₂S donor added together with Gm was as effective as dipyridyl or thiourea in protecting against antibiotics, but failed to further protect cells that had already been pretreated with antioxidants (FIG. 10A). Thus, H₂S, like NO, acts by suppressing the oxidative component of antibiotic toxicity.

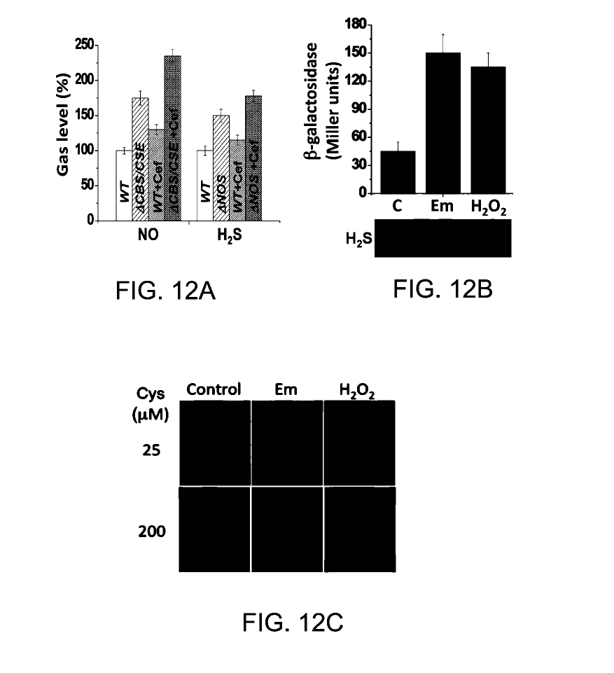

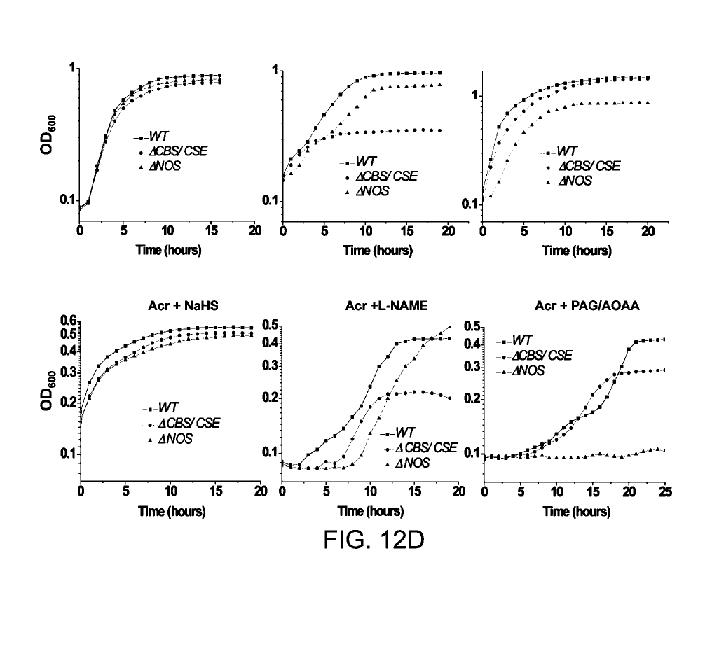

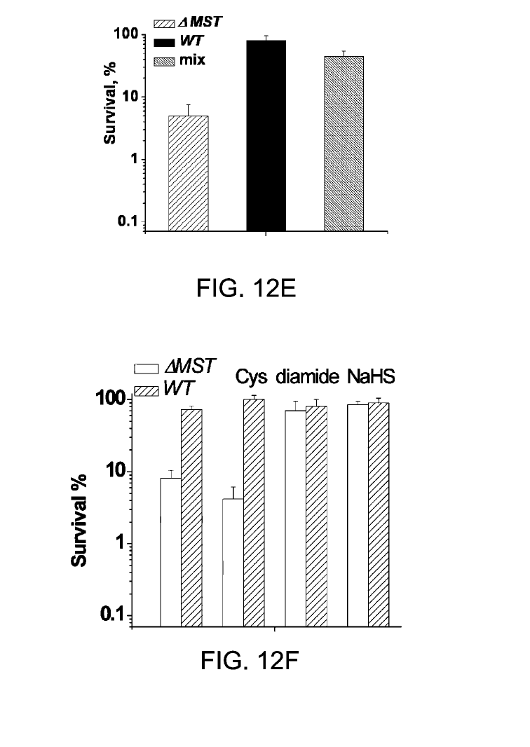

Consistently, H₂S-generating enzymes provided protection against antibiotics only under aerobic conditions. As shown in FIGS. 10B and 10C, nalidixic acids (NA) and pyocyanin, respectively, were considerably less potent against CBS/CSE-deficient B. anthracis in anaerobic growth conditions than in aerobic conditions. Such a substantial difference supports the notion that these antibiotics kill bacteria in large part by causing oxidative stress (I. Gusarov, K. Shatalin, M. Starodubtseva, E. Nudler, Science 325, 1380 (2009); M. A. Kohanski, D. J. Dwyer, B. Hayete, C. A. Lawrence, J. J. Collins, Cell 130, 797 (2007); and M. A. Kohanski, D. J. Dwyer, J. J. Collins, Nat. Rev. Microbiol. 8, 423 (2010)). Because H₂S completely fails to protect against NA in anaerobic conditions, it can be concluded that, at least in the case of this quinolone, all the protective effect of H₂S can be attributed to its ability to mitigate the oxidative damage caused by the antibiotic. These results also suggest that the effectiveness of many antibiotics whose potencies have been associated with oxidative stress (I. Gusarov, K. Shatalin, M. Starodubtseva, E. Nudler, Science 325, 1380 (2009); and M. A. Kohanski, D. J. Dwyer, B. Hayete, C. A. Lawrence, J. J. Collins, Cell 130, 797 (2007)) would be compromised during infections caused by anaerobic bacteria such as Bacteroides or Clostridium. It remains to be determined to what extent H₂S production by bacteria during aerobic and anaerobic infection contributes to antibiotic resistance. However, considering the parallels between NO and H₂S production by bacteria (FIG. 12) and the essential role of bacterial NO in survival of germinating B. anthracis in macrophages (K. Shatalin et al., Proc. Natl. Acad. Sci. U.S.A. 105, 1009 (2008)), it is possible that H₂S-mediated protection against oxidative stress contributes to bacterial infection and antibiotic resistance as well.

The above results suggested that H₂S bolsters the antioxidant capacity of bacterial cells. Indeed, H₂S-deficient B. anthracis, E. coli, S. aureus, and P. aeruginosa displayed higher susceptibility to peroxide than their wt counterparts, whereas NaHS rendered them more resistant to the peroxide (FIGS. 10D-10G).

Formation of double-strandDNAbreaks (DSBs) is the primary cause of bacterial death from peroxide (O. I. Aruoma, B. Halliwell, E. Gajewski, M. Dizdaroglu, J. Biol. Chem. 264, 20509 (1989); and S. I. Liochev, I. Fridovich, IUBMB Life 48, 157 (1999)). These DSBs result from the Fenton reaction (J. A. Imlay, Annu. Rev. Microbiol. 57, 395 (2003)), which also can be triggered by antibiotics (I. Gusarov, K. Shatalin, M. Starodubtseva, E. Nudler, Science 325, 1380 (2009); M. A. Kdhanski, D. J. Dwyer, B. Hayete, C. A. Lawrence, J. J. Collins, Cell 130, 797 (2007); M. A. Kohanski, J. Dwyer, J. J. Collins, Nat. Rev. Microbiol. 8, 423 (2010); and B. W. Davies et al., Mol. Cell. 36, 845 (2009)). To examine whether H₂S protects bacteria from the damaging Fenton reaction, chromosomal DNA integrity was monitored by pulsed-field gel electrophoresis (PFGE) (FIG. 10H). The intact E. coli chromosome does not migrate into the agarose gel but remains at the origin (B. Birren, E. Lai, Pulsed-Field Gel Electrophoresis: A Practical Guide (Academic Press, New York, 1993)), whereas linear chromosomes containing a single DSB migrate as a 4.6-Mb species (FIG. 10H, lane 1). Absent antibiotic or H₂O₂, DNA isolated from wt or H₂S-deficient cells was retained almost entirely at the origin (lanes 2 and 3). However, treatment of cells with a sublethal dose of H₂O₂ or ampicillin resulted in a greater linearization (DSBs) of the chromosome in 3MST-deficient cells (lanes 4 and 6). Overexpression of 3MST suppressed this linearization (lane 8), as did treatment with NaHS (lanes 9 and 10). These results were corroborated by polymerase chain reaction (PCR) analysis of B. anthracis, E. coli, and P. aeruginosa genomic DSBs as a function of H₂S production (FIG. 10I) and further supported by the ability of H₂S to suppress the Gm-induced SOS response (FIG. 10J). Taken together, these results directly implicate endogenous H₂S in the mitigation of chromosomal damage inflicted by antibiotics.

The antioxidant effect of endogenous H₂S can also be explained, in part, by its ability to augment the activities of catalase and superoxide dismutase (SOD) (FIG. 10K). The rate of H₂O₂ degradation in crude extracts of wt E. coli cells was >1.5 times that of 3MST-deficient cells and was increased further in cells that overexpressed 3MST (FIG. 10K). SOD activity also was proportional to the level of 3MST expression (FIG. 10K).

The extent of genomic DSBs can be assessed by PCR of an arbitrarily chosen segment of the bacterial chromosome (I. Gusarov, E. Nudler, Proc. Natl. Acad. Sci. U.S.A. 102, 13855 (2005)). In this assay, equal amounts of genomic DNA from wt and mutant cells grown with or without antibiotics or H₂O₂ were isolated for PCR. The number of reaction cycles was selected so that DSBs influenced the yield of the final PCR product. The yield of the PCR product from exponentially growing, H₂S-deficient B. anthracis, E. coli, or P. aeruginosa treated with H₂O₂ or antibiotics was substantially lower than that from similarly treated wt cells (FIG. 10I).

To further implicate H₂S in genome maintenance, its effect on the SOS response was monitored using a chromosomal recA-gfp gene fusion (M. Kostrzynska, K. T. Leung, H. Lee, J. T. Trevors, J. Microbiol. Methods 48, 43 (2002)). Addition of a sub-lethal amount of gentamicin induced significant GFP fluorescence (FIG. 10J). However, induction of RecA by the antibiotic was more pronounced in aspartate-treated (3MST-compromised) cells than in untreated cells (FIG. 10J). NaHS completely suppressed the hyper-induction of SOS by gentamicin.

Thus, H₂S increases bacteria resistance to oxidative stress and antibiotics by a dual mechanism (FIG. 10L) of suppressing the DNA-damaging Fenton reaction via Fe²⁺ sequestration (FIGS. 10A, 10F, and 10M) and stimulating the major antioxidant enzymes catalase and SOD (FIG. 10K). The latter is essential for long-term protection but is less important during the first moments of oxidative stress. Indeed, katE and sodA E. coli mutants are well protected by NaHS during the first minutes of H₂O₂ exposure but then quickly lose viability (FIG. 10N).

Taken together, these results directly implicate endogenous H₂S in the mitigation of chromosomal damage inflicted by antibiotics, and indicate that endogenous H₂S renders bacteria more resistant to oxidative stress and antibiotics by suppressing the DNA-damaging Fenton reaction.

### Example 5

### Endogenous H₂S and NO Act Synergistically

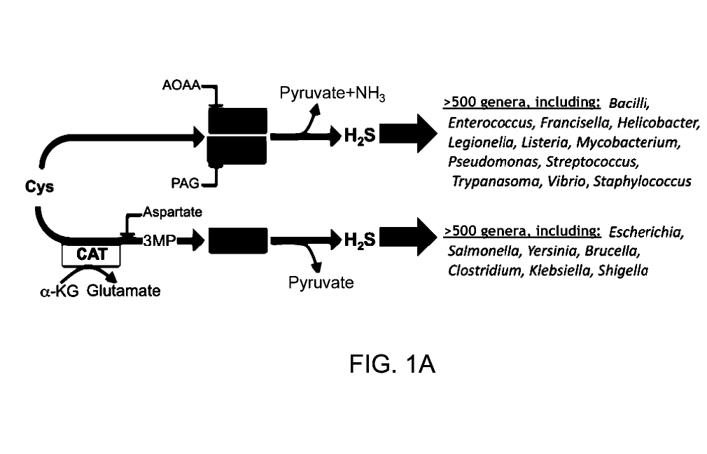

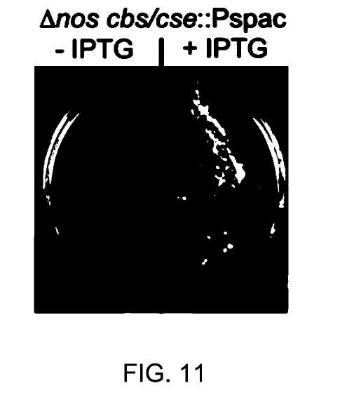

This cytoprotective mechanism of H₂S parallels that of NO (I. Gusarov, K. Shatalin, M. Starodubtseva, E. Nudler, Science 325, 1380 (2009)), which suggests that bacteria that produce both gases may benefit from their synergistic action. To test this hypothesis, the effect of simultaneously inhibiting H₂S and NO on B. anthracis growth was examined. A strain of B. anthracis in which both bacterial nitric oxide synthase (bNOS) and CBS/CSE were genetically inactivated could not be generated, suggesting that the absence of both gases is incompatible with B. anthracis survival. Indeed, B. anthracis Δnos cells containing an isopropylb-D-thiogalactopyranoside (IPTG)-inducible CBS/CSE conditional knockout could grow only in the presence of IPTG (FIG. 11). Notably, the amount of NO produced in H₂S-deficient cells or the amount of H₂S produced in NO-deficient cells was greater than that produced in wt cells (FIG. 12A), which indicated that one gas compensates for the lack of the other. Also, the activity of both CBS/CSE and bNOS was stimulated in response to antibiotics (FIG. 12A). Moreover, H₂O₂ and antibiotics (e.g., erythromycin) substantially induced CBS/CSE gene expression (FIG. 12B) and H₂S production (FIGS. 12B and 12C). Furthermore, chemical inhibition of CBS/CSE in bNOS-deficient cells or inhibition of bNOS in CBS/CSE-deficient cells sensitized B. anthracis to antibiotics to a much greater extent than did each mutation alone (FIG. 12C). These results in concert with previous results (I. Gusarov, K. Shatalin, M. Starodubtseva, E. Nudler, Science 325, 1380 (2009)) demonstrate the synergistic and specific protective effects of H₂S and NO against antibiotics. Notably, in contrast to bNOS, which is present in only a small number of Gram-positive species (I. Gusarov et al., J. Biol. Chem. 283, 13140 (2008)), H₂S enzymes are essentially universal (FIG. 1A). Because H₂S equilibrates rapidly across cell membranes, a fraction of cells that generate this gas in culture or in biofilms could, in principle, defend the entire population. Indeed, wt E. coli cells effectively protect 3MST-deficient cells from Gm toxicity in exponentially growing coculture (FIG. 12E).

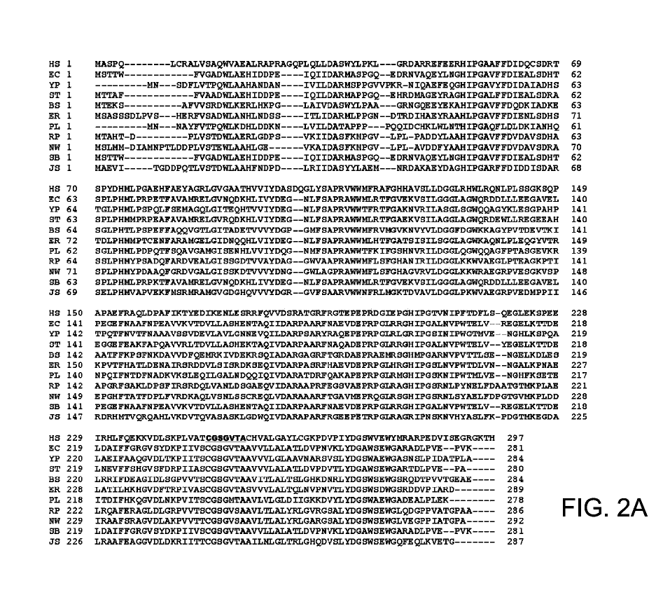

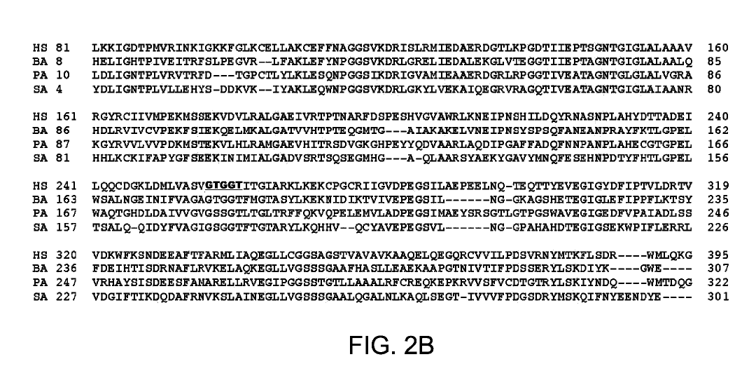

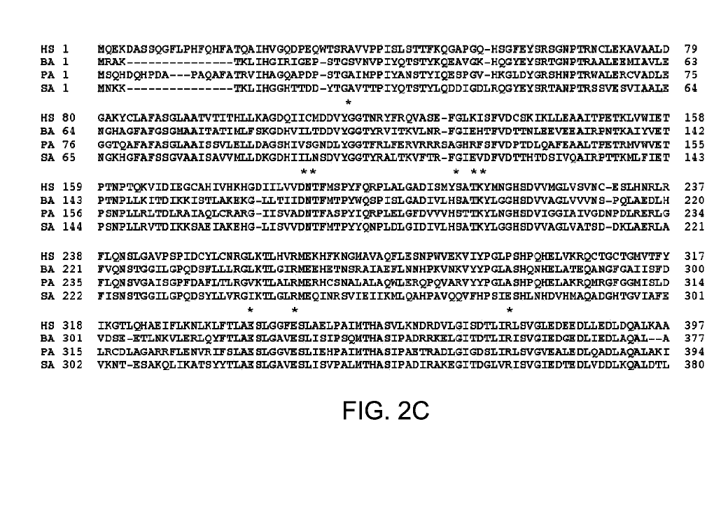

Because endogenous H₂S diminishes the effectiveness of many clinically used antibiotics, the inhibition of this “gaskeeper” should be considered as an augmentation therapy against a broad range of pathogens. Bacterial CBS, CSE, and 3MST have diverged substantially from their mammalian counterparts (FIG. 2), suggesting that it is possible to design specific inhibitors targeting these enzymes.

TABLE 1
List of chemicals that selectively inhibit the growth of E. coli
ΔsseA (3MST-deficient) strain. The results are provided by
Phenotype MicroArray (Biolog, Inc). The relative values of
growth inhibition (negative numbers) are presented in the
table (see FIG. 8 for raw data).

| | | ΔsseA | |
|---|---|---|---|
| | | inhibition | |
| Map position | | of growth | Chemical |
| PM12B | D09, D10, D12 | −265 | Novobiocin |
| PM16A | B01, B02 | −212 | Norfloxacin |
| PM11C | E09, E10 | −183 | Nalidixic acid |
| PM13B | B11 | −117 | Oxolinic acid |
| PM14A | A01, A02, A03 | −295 | Acriflavine |
| PM14A | B02, B03 | −142 | 9-Aminoacridine |
| PM20B | D03 | −93 | Proflavine |
| PM13B | G09, G10, G12 | −372 | Trifluoperazine |
| PM14A | H05, H06 | −262 | Promethazine |
| PM17A | D09, D10 | −251 | Chlorpromazine |
| PM16A | E01, E02, E03, E04 | −421 | Streptomycin |
| PM20B | A05, A06 | −179 | Apramycin |
| PM13B | H09, H10, H11, H12 | −520 | Tylosin |
| PM15B | F05, F06, F07 | −498 | Oleandomycin |
| PM11C | F05, F06, F07, F08 | −468 | Erythromycin |
| PM19 | A01, A02 | −294 | Josamycin |
| PM12B | H01, H02 | −235 | Spiramycin |
| PM20B | H09, H10 | −203 | Troleandomycin |
| PM11C | F01, F02 | −136 | Chloramphenicol |
| PM15B | C05, C06 | −271 | Fusidic acid |
| PM16A | E09, E10, E11, E12 | −352 | Rifamycin SV |
| PM16A | B09, B10 | −212 | Trimethoprim |
| PM12B | E01, E02, E03 | −429 | 2,4-Diamino-6,7- |
| | | | diisopropylpteridine |
| PM12B | B09, B10, B11 | −231 | Polymyxin B |
| PM11C | C06 | −102 | Colistin |
| PM12B | C05, C06 | −198 | Vancomycin |
| PM17A | H01, H02, H03, H04 | −201 | Cefsulodin |
| PM11C | H01, H02 | −160 | Cephalothin |
| PM17A | G09, G10 | −159 | Cefoperazone |
| PM12B | B01, B02 | −346 | Oxacillin |
| PM11C | D09, D10 | −227 | Nafcillin |
| PM19 | F01, F02 | −216 | Phenethicillin |
| PM12B | A01, A02 | −186 | Penicillin G |
| PM11C | B05, B06 | −182 | Cloxacillin |
| PM13B | H06, H07 | −171 | Moxalactam |
| PM12B | A09, A10 | −164 | Carbenicillin |
| PM20B | A10, A11, A12 | −336 | Benserazide |
| PM17A | G02, G03, G04 | −177 | Chlorambucil |
| PM20B | B01, B02 | −345 | Orphenadrine |
| PM20B | F09, F10 | −206 | Pridinol |
| PM20B | B05, B06 | −263 | Propranolol |
| PM18C | A01, A02, A03, A04 | −468 | Ketoprofen |
| PM17A | H06, H07 | −173 | Caffeine |
| PM19 | G09, G10 | −150 | Hydroxylamine |
| PM19 | A09, A10, A11 | −243 | Coumarin |
| PM17A | A01, A02, A03 | −398 | D-Serine |
| PM18C | D09, D10 | −269 | Lidocaine |
| PM18C | C01, C02, C03 | −377 | Poly-L-lysine |
| PM19 | B01, B02 | −326 | Methyltrioctylammonium |
| | | | chloride |
| PM16A | C09, C10 | −269 | Cetylpyridinium chloride |
| PM12B | E09, E10 | −255 | Benzethonium chloride |
| PM12B | H09, H10 | −225 | Dodecyltrimethyl |
| | | | ammonium bromide |
| PM15B | D05, D06 | −250 | Domiphen bromide |
| PM15B | E01, E02 | −216 | Alexidine |
| PM20B | A01, A02, A03 | −422 | Amitriptyline |
| PM20B | G01, G02, G03 | −357 | Captan |
| PM18C | F06, F07 | −223 | Tinidazole |
| PM20B | C09, C10 | −203 | Ornidazole |
| PM14A | A05, A06 | −162 | Furaltadone |
| PM18C | B09, B10, B11, B12 | −341 | Azathioprine |
| PM13B | E01, E02, E03 | −281 | Cytosine arabinoside |
| PM19 | D01, D02 | −246 | Disulfiram |
| PM09 | G01, G02, G03, G04 | −497 | 200 mM Sodium |
| | | | Phosphate pH 7 |
| PM16A | D01, D02 | −221 | 1-Chloro-2,4- |
| | | | dinitrobenzene |
| PM19 | E09, E10, E11, E12 | −330 | Lawsone |
| PM19 | D09, D10 | −194 | Phenyl-methylsulfonyl- |
| | | | fluoride (PMSF) |
| PM19 | F06, F07 | −185 | Blasticidin S |
| PM19 | D05, D06 | −215 | Iodonitro tetrazolium violet |
| PM19 | E01, E02, E03 | −460 | FCCP |
| PM15B | G01, G02, G03, G04 | −422 | CCCP |
| PM19 | F09, F10, F11, F12 | −346 | Sodium caprylate |
| PM19 | B09, B10, B11 | −296 | 2,4-Dinitrophenol |
| PM18C | C09, C10, C11 | −287 | Pentachlorophenol |
| PM20B | D09, D10, D11 | −212 | 18-Crown-6 ether |
| PM19 | C09, C10 | −204 | Cinnamic acid |
| PM13B | E09, E10, E11 | −268 | Ruthenium red |
| PM20B | E01, E02, E03, E04 | −576 | Crystal Violet |
| PM20B | C01, C02, C03 | −313 | Thioridazine |
| PM20B | B09, B10 | −293 | Tetrazolium violet |
| PM14A | C09, C10, C11 | −238 | Sodium cyanate |

TABLE 2
3MST protects E. coli against different classes of antibiotics

| ΔsseA | | |
|---|---|---|
| inhibition | Chemical | Biological effect/type |
| −265 | Novobiocin | DNA intercalation/quinolone |
| −212 | Norfloxacin | DNA intercalation/quinolone |
| −183 | Nalidixic acid | DNA intercalation/quinolone |
| −117 | Oxolinic acid | DNA intercalation/quinolone |
| −295 | Acriflavine | DNA intercalation/acridine |
| −142 | 9-Aminoacridine | DNA intercalation/acridine |
| −93 | Proflavine | DNA intercalation/acridine |
| −372 | Trifluoperazine | DNA intercalation/phenothiazine |
| −262 | Promethazine | DNA intercalation/phenothiazine |
| −251 | Chlorpromazine | DNA intercalation/phenothiazine |
| −421 | Streptomycin | Protein synthesis/aminoglycoside |
| −179 | Apramycin | Protein synthesis/aminoglycoside |
| −520 | Tylosin | Protein synthesis/macrolide |
| −498 | Oleandomycin | Protein synthesis/macrolide |
| −468 | Erythromycin | Protein synthesis/macrolide |
| −294 | Josamycin | Protein synthesis/macrolide |
| −235 | Spiramycin | Protein synthesis/macrolide |
| −203 | Troleandomycin | Protein synthesis/macrolide |
| −136 | Chloramphenicol | Protein synthesis/amphenicol |
| −271 | Fusidic acid | Protein synthesis/steroid |
| −352 | Rifamycin SV | RNA synthesis/ansamycin |
| −212 | Trimethoprim | DNA/RNA synthesis/antifolate |
| −429 | 2,4-Diamino-6,7- | DNA/RNA synthesis/antifolate |
| | diisopropylpteridine | |
| −231 | Polymyxin B | Membrane/polymyxin |
| −102 | Colistin | Membrane/polymyxin |
| −198 | Vancomycin | Cell wall/glycopeptide |
| −201 | Cefsulodin | Cell wall/cephalosporin |
| −160 | Cephalothin | Cell wall/cephalosporin |
| −159 | Cefoperazone | Cell wall/cephalosporin |
| −346 | Oxacillin | Cell wall/lactam |
| −227 | Nafcillin | Cell wall/lactam |
| −216 | Phenethicillin | Cell wall/lactam |
| −186 | Penicillin G | Cell wall/lactam |
| −182 | Cloxacillin | Cell wall/lactam |
| −171 | Moxalactam | Cell wall/lactam |
| −164 | Carbenicillin | Cell wall/lactam |

### Example 6

### The Mechanism of H₂S-Mediated Cytoprotection

The results presented herein suggest several non-mutually exclusive mechanisms that could explain the H2S-mediated defense against oxidative stress (FIG. 10L): (i) stimulation of the major antioxidative enzymes, catalase and SOD (FIG. 10K); (ii) direct neutralization of peroxide (H₂S+H₂O₂→S+2H₂O); (iii) transient depletion of free cysteine, a reducing agent that fuels the Fenton reaction (S. Park, J. A. Imlay, J. Bacteriol. 185, 1942 (2003)), and (iv) depletion of ferrous iron, a catalytic agent of the Fenton reaction. It is possible that the sequestration of ferrous iron by H₂S is the largest contributor to H₂S-mediated protection against oxidative stress (although other mechanisms play an important role as well, as described below). To test this hypothesis, the effect of NaHS on cell survival was measured after treatment with a lethal dose of H₂O₂. Within one minute of pretreatment with NaHS, cells became >20 times more resistant to peroxide (FIG. 10F). This rules out any protective mechanism that depends on gene expression. It is noted that the amount of NaHS was ten times smaller than that of peroxide, i.e., the protective effect could not be explained by direct reaction of NaHS with peroxide in this case. Furthermore, pretreatment of cells with the Fe chelator dipyridyl also protected them rapidly against peroxide (FIG. 10F). Because NaHS does not provide extra protection beyond that of dipyridyl (FIG. 10F), it can be concluded that, like dipyridyl, NaHS protected cells from the Fenton reaction by eliminating Fe⁺⁺.

To further investigate the validity of this conclusion, the effect of H₂S on the Fenton reaction in vitro was examined. Hydrogen peroxide alone or together with Fe³⁺ did not produce significant strand brakes in pBR322DNA (FIG. 10M), whereas addition of Cys along with Fe³⁺ caused dramatically accelerated DNA damage. Addition of NaHS (NaHS:Fe³⁺-6:1) completely inhibited DNA damage under physiological pH (pH=8). It is noted that NaHS was 20 times less abundant than H₂O₂. This result shows that H₂S can effectively interfere with Fenton chemistry by scavenging cellular iron.

To examine the role of free Cys in oxidative stress associated with antibiotics, its effect on gentamicin (Gm) toxicity was studied in wt and 3MST-deficient cells. WT E. coli became more resistant to the antibiotic in Cys-enriched media (0.5 mM L-cysteine) than in regular LB (<20 μM L-cysteine) (FIG. 12F). In contrast, 3MST-deficient cells became more sensitive to Gm in Cys rich media. This stronger sensitivity of the 3MST mutant cells to Gm killing in the presence of Cys was attributed to the higher level of intercellular pro-oxidative Cys that could not be efficiently converted to protective H₂S. Indeed, addition of the thiol-depleting diamide protected 3MST mutant cells against Gm (FIG. 12F).

### Example 7

### Antibiotic Potentiation by AOAA Against Enterococcus faecalis

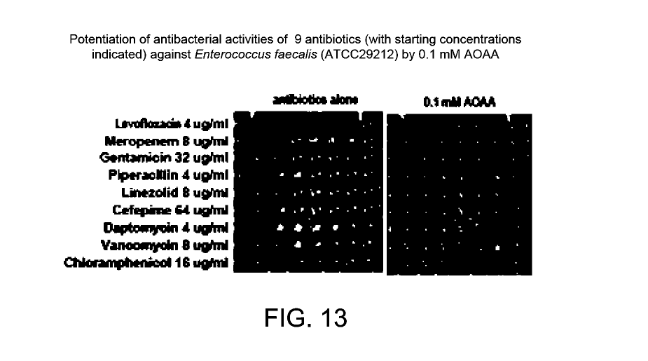

A study was conducted to demonstrate potentiation of antibiotics by AOAA. E. faecalis (ATCC 29212) cultures were prepared at initial inoculums of ˜5×10⁵ CFU/ml in Mueller-Hinton II Broth (cation-adjusted). Aliquots of 190 μl were dispensed to each well of a fresh set of plates, and 10 μl of an antibiotic solution (levofloxacin, meropenem, gentamicin, piperacillin, linezolid, cefepime, daptomycin, vancomycin, or chloramphemicol) were transferred to the corresponding wells in each bacterial plate (FIG. 13). For each antibiotic, the starting concentration was 2×MIC (minimum inhibitory concentration) with serial ½ dilutions. Two sets of dilutions were prepared, and to one of them 0.1 mM AOAA was added. The resulting plates were kept at 37° C. and 85% humidity for 20 hours. All nine antibiotics tested were potentiated by AOAA, with respective MICs dropped from 8-fold to 32-fold (FIG. 13). Thus, this study demonstrated that the activities of the antibiotics tested were potentiated by AOAA.

### Example 8

### Comparison of Antibiotic Potentiation by AOAA and PAG

Minimum inhibitory concentration (MIC) determination was conducted as described in Example 7, above. To assess antibiotic potentiation by AOAA and PAG, MIC was determined for nine antibiotics (levofloxacin, meropenem, gentamicin, piperacillin, linezolid, cefepime, daptomycin, vancomycin, and chloramphemicol) and four bacterial strains (E. faecium (A2373), E. faecalis (ATCC 9212), S. pneumoniae (ATCC 49619), and P. aeruginosa (PAO1)) in the presence of antibiotic alone, 1 mM PAG, 0.1 mM AOAA, or 0.5 mM PAG+0.05 mM AOAA. The results are summarized in Table 3. The smaller the MIC value, the stronger the effect of antibiotic potentiation. As shown in Table 3, in most cases, AOAA has a stronger antibiotic potentiation effect than PAG or even AOAA+PAG. These data also suggest that inhibiting CBS (targeted by AOAA) may be more clinically relevant than inhibiting CSE (targeted by PAG) (FIG. 1A).

TABLE 3
Minimum inhibitory concentrations (in μg/ml) of antibiotics in the presence
of antibiotic alone, 1 mM PAG, 0.1 mM AOAA, or 0.5 mM PAG + 0.05 mM AOAA.

| | E. faecium A2373 | | | | E. faecalis ATCC29212 | | | |
|---|---|---|---|---|---|---|---|---|
| Antibiotics/ | | | | PAG + | | | | PAG + |
| compounds | lone | PAG | AOAA | AOAA | alone | PAG | AOAA | AOAA |
| Levoflaxacin | 2 | 2 | 2 | 2 | 1 | 1 | 0.125 | 0.5 |
| Meropenom | >64 | >64 | 6 | >64 | 4 | 4 | 0.25 | 1 |
| Gentamicin | >64 | >64 | >64 | >64 | 32 | 32 | 1 | 4 |
| Piperacilin | >64 | >64 | 6 | >64 | 2 | 2 | 0.25 | 1 |
| Linezold | 4 | 4 | 2 | 4 | 4 | 4 | 0.25 | 2 |
| Cetepine | >64 | >64 | >64 | >64 | 32 | 32 | 2 | 8 |
| Daptomycin | 4 | 2 | ≦1 | 4 | 2 | 2 | ≦0.06 | 1 |
| Vancomycin | >64 | >64 | 2 | >64 | 2 | 2 | 1 | 2 |
| Chloramphenicol | 8 | 8 | 2 | 8 | 8 | 8 | 0.5 | 8 |
| | S. pneumoniae ATCC49619 | | | | P. aeruginosa PAO1 | | | |
| Antibiotics/ | | | | PAG + | | | | PAG + |
| compounds | alone | PAG | AOAA | AOAA | alone | PAG | AOAA | AOAA |
| Levoflaxacin | 2 | 1 | 0.5 | 0.5 | 0.5 | 0.5 | 0.5 | 0.5 |
| Meropenom | 0.125 | 0.063 | 0.063 | 0.063 | 1 | 0.5 | 0.5 | 0.5 |
| Gentamicin | 32 | 32 | 8 | 16 | 2 | 2 | 2 | 4 |
| Piperacilin | 0.5 | 0.5 | 0.25 | 0.5 | 4 | 4 | 4 | 4 |
| Linezold | 2 | 2 | 0.5 | 1 | >64 | >64 | >64 | >64 |
| Cetepine | 0.125 | 0.125 | 0.125 | 0.125 | 2 | 2 | 2 | 1 |
| Daptomycin | 0.125 | 0.125 | 0.063 | 0.125 | >64 | >64 | >64 | >64 |
| Vancomycin | 0.125 | 0.125 | 0.125 | 0.125 | >64 | >64 | >64 | >64 |
| Chloramphenicol | 8 | 4 | 4 | 4 | 64 | 64 | 64 | 64 |

### REFERENCES

1. H. Kimura, Antioxid Redox Signal. 12, 1111-1123 (2010).
2. G. Yang, et al. Science 322, 587-590 (2008).
3. Q. C. Yong, S. W. Lee, C. S. Foo, K. L. Neo, X. Chen, J. S. Bian. Am J Physiol Heart Circ Physiol 295, H1330-H1340 (2008).
4. M. M. Gadalla, S. H. Snyder, J. Neurochem. 113, 14-26 (2010).
5. D. R. Linden, M. D. Levitt, G. Farrugia, J. H. Szurszewski, Antioxid Redox Signal. 12, 1135-1146 (2010).
6. R. Wang, Antioxid Redox Signal. 12, 1061-1064 (2010).
7. A. K. Mustafa, M. M. Gadalla, S. H. Snyder, Sci Signal. 2, re2 (2009).
8. B. Lima, M. T. Forrester, D. T. Hess, J. S. Stamler, Circ Res. 106, 633-646 (2010).
9. M. Ishigami, K. Hiraki, K. Umemura, Y. Ogasawara, K. Ishii, H. Kimura, Antioxid Redox Signal. 11, 205-214 (2009).
10. I. Gusarov, E. Nudler, Proc Natl Acad Sci USA 102, 13855-60 (2005).
11. K. Shatalin et al., Proc Natl Acad Sci USA 105, 1009-13 (2008).
12. I. Gusarov, K. Shatalin, M. Starodubtseva, E. Nudler, Science 325, 1380-1384 (2009).
13. B. A. Forbes, Bailey and Scott's Diagnostic Microbiology, 10th ed. C. V. Mosby Company, St. Louis, Mo. (1998).
14. R. Lutz, H. Bujard, Nucl Acid Res. 25, 1203-1210 (1997).
15. M. A. Kohanski, D. J. Dwyer, B. Hayete, C. A. Lawrence, J. J. Collins, Cell 130, 797-810 (2007).
16. N. R. Asad, A. C. Leitao, J Bacteriol 173, 2562-2568 (1991).
17. O. I. Aruoma, B. Halliwell, E. Gajewski, M. Dizdaroglu J Biol Chem. 264, 20509-20512 (1989).
18. S. I. Liochev, I. Fridovich, IUBMB Life 48, 157-161 (1999).
19. J. A. Imlay, Annu Rev Microbiol. 57, 395-418 (2003).
20. M. A. Kohanski, D. J. Dwyer, J. J. Collins, Nat Rev Microbiol. 8, 423-435 (2010).
21. B. W. Davies, M. A. Kohanski, L. A. Simmons, J. A. Winkler, J. J. Collins, G. C. Walker, Mol Cell 36, 845-860 (2009).
22. B. Birren, E. Lai, Pulse Field Gel Electrophoresis, a Practical Guide (Academic Press, New York; 1993).
23. A. Azvolinsky, S. Dunaway, J. Z. Tones, J. B. Bessler, V. A. Zakian, Genes Dev 20, 3104-311 (2006).
24. M. Kostrzynska, K. T. Leung, H. Lee, J. T. Trevors, J Microbiol Methods 48, 43-51 (2002).
25. U. Gayon, Comp Rend Acad Sci 85: 1074-1076, 1877.
26. P. Bruinenberg, G. De Roo, G. Limsowtin, Appl Environ Microbiol. 63, 561-566 (1997).
27. W.-J. Lee, et al., Appl Environ Microbiol. 73, 3034-3039 (2007).
28. M. H. Stipanuk, P. W. Beck, Biochem J. 206, 267-277 (1982).
29. Y. J. Peng et al., Proc Natl Acad Sci USA. 107, 10719-10724 (2010).
30. S. Park, J. A. Imlay, J. Bacteriol. 185, 1942-1950 (2003).
31. O. Kabil, R. Banerjee J Biol Chem. 285, 21903-21907 (2010).
32. I. Gusarov, et al, J Biol Chem. 283, 13140-13147 (2008).
33. M. H. Lim, D. Xu, S. J. Lippard, Nat Chem Biol 2, 375-380 (2006).

### SUPPLEMENTAL REFERENCES

S1. K. Datsenko, B. Wanner, Proc Natl Acad Sci USA 97, 6640-45 (2000).
S2. J. Katashkina et al., Mol Biol (Mosk) 39, 823-31 (2005).
S3. R. Lutz, H. Bujard, NAR 25, 1203-10 (1997).
S4. K. Shatalin et al., Proc Natl Acad Sci USA 105, 1009-13 (2008).
S5. K. Shatalin, A. Neyfakh, FEMS Microbiol. Lett. 245, 315-19 (2005).
S6. B. Forbes et al., Bailey and Scott's Diagnostic Microbiology, 10th ed. C. V. Mosby Company, St. Louis, Mo. (1998)
S7. M. Kdstrzynska et al., J Microbiol Methods 48, 43-51 (2002).
S8. L. Chen et al., Proc Natl Acad Sci USA 92, 8190-4 (1995).
S9. I. Dunham et al., Nature 402, 489-95 (1999).
S10. A. Spallarossa et al., Acta Crist. D59, 168-70 (2003).
S11. R. Strausberg et al., Proc Natl Acad Sci USA 99, 16899-903 (2002).
S12. S. Gregory et al., Nature 441, 315-21 (2006).
S13. M. Meier et al., EMBO Journal 20, 3910-16 (2001).
S14. Q. Sun et al., J. Biol. Chem. 284, 3076-85 (2009).

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

### LIST OF SEQUENCES

(SEQ ID NO: 1)
5′-atgcgtgagaatttacgttatgtaattcagtatcaccgctcaagtt
agtataaaaaagct-3′
(SEQ ID NO: 2)
5′-ttatttcactggctcaaccggtaaatctgcccgcgtgaagcctgct
tttttatactaag-3′
(SEQ ID NO: 3)
5′-attagcatgcaggcggctac-3′
(SEQ ID NO: 4)
5′-tgctggaaaggccgcgaa-3′
(SEQ ID NO: 5)
5′-cgcagatcttgaagcctgcttttttatac-3′
(SEQ ID NO: 6)
5′-cgctcaagttagtataaaaaagct-3′
(SEQ ID NO: 7)
5′-cgcagatctcgagtccctatcagtg-3′
(SEQ ID NO: 8)
5′-aatttctcctctttccatgg-3′
(SEQ ID NO: 9)
5′-ccctgccacaatggcccgttagcaacgtcgaataacgctcaagtta
gtataaaaaagct-3′
(SEQ ID NO: 10)
5′-gtgatactgaattacataacgtaaattctcacgcatggtacctttc
tcctctttaatga-3′
(SEQ ID NO: 11)
5′-attagcatgcaggcggctac-3′
(SEQ ID NO: 12)
5′-gcaaaagaggctgatttggct-3′
(SEQ ID NO: 13)
5′-ttccattgggatgtagatgctg-3′
(SEQ ID NO: 14)
5′-ggtaaaagagtcaagggaagaacc-3′
(SEQ ID NO: 15)
5′-acgctttgacttctctcacttcggt-3′
(SEQ ID NO: 16)
5′-aaacatttgctcttgatgtcctgga-3′
(SEQ ID NO: 17)
5′-ctttgcaacctgtacatgccttg-3′
(SEQ ID NO: 18)
5′-catcgtagtagttgatcggatggac-3′
(SEQ ID NO: 19)
5′-ggggacaagtttgtacaaaaaagcaggctaagggggagaacacgat
gaatg-3′
(SEQ ID NO: 20)
5′-ggggaccactttgtacaagaaagctgggtgtgccgaccaaagttca
ggac-3′
(SEQ ID NO: 21)
MASPQLCRALVSAQWVAEALRAPRAGQPLQLLDASWYLPKLGRDARREF
EERHIPGAAFFDIDQCSDRTSPYDHMLPGAEHFAEYAGRLGVGAATHVV
IYDASDQGLYSAPRVWWMFRAFGHHAVSLLDGGLRHWLRQNLPLSSGKS
QPAPAEFRAQLDPAFIKTYEDIKENLESRRFQVVDSRATGRFRGTEPEP
RDGIEPGHIPGTVNIPFTDFLSQEGLEKSPEEIRHLFQEKKVDLSKPLV
ATCGSGVTACHVALGAYLCGKPDVPIYDGSWVEWYMRARPEDVISEGRG
KTH
(SEQ ID NO: 22)
MSTTWFVGADWLAEHIDDPEIQIIDARMASPGQEDRNVAQEYLNGHIPG
AVFFDIEALSDHTSPLPHMLPRPETFAVAMRELGVNQDKHLIVYDEGNL
FSAPRAWWMLRTFGVEKVSILGGGLAGWQRDDLLLEEGAVELPEGEFNA
AFNPEAVVKVTDVLLASHENTAQIIDARPAARFNAEVDEPRPGLRRGHI
PGALNVPWTELVREGELKTTDELDAIFFGRGVSYDKPIIVSCGSGVTAA
VVLLALATLDVPNVKLYDGAWSEWGARADLPVEPVK
(SEQ ID NO: 23)
MNSDFLVTPQWLAAHANDANIVILDARMSPPGVVPKRNIQAEFEQGHIP
GAVYFDIDAIADHSTGLPHMLPSPQLFSEMAGQLGITEQHTVVIYDEGN
LFSAPRVWWTFRTFGAKNVRILASGLSGWQQAGYKLESGPAHPTPQTFN
VTFNAAAVSSVDEVLAVLGNNEVQILDARPSARYRAQEPEPRPGLRLGR
IPGSINIPWGTMVENGHLKSPQALAEIFAAQGVDLTKPIITSCGSGVTA
AVVVLGLAAVNARSVSLYDGSWAEWGASNSLPIDATPLA
(SEQ ID NO: 24)
MTTAFFVAADWLAEHIDDPEIQILDARMAPPGQEHRDMAGEYRAGHIPG
ALFFDIEALSDRASPLPHMMPRPEAFAVAMRELGVRQDKHLVIYDEGNL
FSAPRAWWMLRTFGAEKVSILAGGLAGWQRDEWLLREGEEAHEGGEFEA
KFAPQAVVRLTDVLLASHEKTAQIVDARPAARFNAQADEPRPGLRRGHI
PGALNVPWTELVYEGELKTTDELNEVFFSHGVSFDRPIIASCGSGVTAA
VVVLALATLDVPDVTLYDGAWSEWGARTDLPVEPA
(SEQ ID NO: 25)
MTEKSAFVVSRDWLKERLHKPGLAIVDASWYLPAAGRNGQEEYEKAHIP
GAVFFDQDKIADKESGLPHTLPSPEFFAQQVGTLGITADETVVVYDGPG
MFSAPRVWWMFRVMGVKNVYVLDGGFDGWKKAGYPVTDEVTKIAATFFK
PSFNKDAVVDFQEMRKIVDEKRSQIADARGAGRFTGRDAEPRAEMRSGH
MPGARNVPVTTLSENGELKDLESLRRIFDEAGIDLSGPVVTSCGSGVTA
AVITLALTSLGHKDNRLYDGSWSEWGSRQDTPVVTGEAE
(SEQ ID NO: 26)
MSASSSDLPVSHERFVSADWLANHLNDSSITLIDARMLPPGNDTRDIHA
EYRAAHLPGAVFFDIENLSDHSTDLPHMMPTCENFARAMGELGIDNQQH
LVIYDEGNLFSAPRAWWMLHTFGATSISILSGGLAGWKAQNLPLEQGYV
TRKPVTFHATLDENAIRSRDDVLSISRDKSEQIVDARPASRFHAEVDEP
RPGLHRGHIPGSLNVPWTDLVNNGALKPNAELATILHKHGVDFTRPIVA
SCGSGVTASVVVLALTQLNVPNVTLYDGSWSDWGSRDDVPIARD
(SEQ ID NO: 27)
MNNAYFVTPQWLKDHLDDKNLVILDATAPPPPQQIDCHKLWLNTHIPGA
QFLDLDKIANHQSGLPHMLPDPQTFSQAVGAMGISENHLVVIYDQGNMF
SAPRAWWTFKIFGSHNVRILDGGLQGWQQAGFPTASGEVKRNPQIFNTD
FNADKVKSLEQILGALNDQQIQIVDARATDRFQAKAPEPRPGLRMGHIP
GSKNIPWTMLVENGHFKSETEITDIFHKQGVDLNKPVITSCGSGMTAAV
LVLGLDIIGKKDVYLYDGSWAEWGADEALPLEK
(SEQ ID NO: 28)
MTAHTDPLVSTDWLAERLGDPSVKIIDASFKMPGVLPLPADDYLAAHIP
GAVFFDVDAVSDHASSLPHMYPSADQFARDVEALGISSGDTVVAYDAGG
WVAAPRAWWMFLSFGHANIRILDGGLKKWVAEGLPTEAGKPTIAPGRFS
AKLDPSFIRSRDQLVANLDSGAEQVIDARAAPRFEGSVAEPRPGLRAGH
IPGSRNLPYNELFDAATGTMKPLAELRQAFERAGLDLGRPVVTSCGSGV
SAAVLTLALYRLGVRGSALYDGSWSEWGLQDGPPVATGPAA
(SEQ ID NO: 29)
MSLMMDIAMNPTLDDPLVSTEWLAAHLGEVKAIDASFKMPGVLPLAVDD
FYAAHIPGAVFFDVDAVSDRASPLPHMYPDAAQFGRDVGALGISSKDTV
VVYDNGGWLAGPRAWWMFLSFGHAGVRVLDGGLKKWRAEGRPVESGKVS
PEPGHFTATFDPLFVRDKAQLVSNLSSCREQLVDARAAARFTGAVMEPR
QGLRSGHIPGSRNLSYAELFDPGTGVMKPLDDIRAAFSRAGVDLAKPVV
TTCGSGVSAAVLTLALYRLGARGSALYDGSWSEWGLVEGPPIATGPA
(SEQ ID NO: 30)
MSTTWFVGADWLAEHIDDPEIQIIDARMASPGQEDRNVAQEYLNGHIPG
AVFFDIEALSDHTSPLPHMLPRPKTFAVAMRELGVNQDKHLIVYDEGNL
FSAPRAWWMLRTFGVEKVSILGGGLAGWQRDDLLLEEGAVELPEGEFNA
AFNPEAVVKVTDVLLASHENTAQIIDARPAARFNAEVDEPRPGLRRGHI
PGALNVPWTELVREGELKTTDELDAIFFGRGVSYDKPIIVSCGSGVTAA
VVLLALATLDVPNVKLYDGAWSEWGARADLPVEPVK
(SEQ ID NO: 31)
MAEVITGDDPQTLVSTDWLAAHFNDPDLRIIDASYYLAEMNRDAKAEYD
AGHIPGARFFDIDDISDARSELPHMVAPVEKFMSRMRAMGVGDGHQVVV
YDGRGVFSAARVWWNFRLMGKTDVAVLDGGLPKWVAEGRPVEDMPPIIR
DRHMTVQRQAHLVKDVTQVASASKLGDWQIVDARAPARFRGEEPETRPG
LRAGRIPNSKNVHYASLFKPDGTMKEGDALRAAFEAGGVDLDKRIITTC
GSGVTAAILMLGLTRLGHQDVSLYDGSWSEWGQFEQLKVETG
(SEQ ID NO: 32)
LKKIGDTPMVRINKIGKKFGLKCELLAKCEFFNAGGSVKDRISLRMIED
AERDGTLKPGDTIIEPTSGNTGIGLALAAAVRGYRCIIVMPEKMSSEKV
DVLRALGAEIVRTPTNARFDSPESHVGVAWRLKNEIPNSHILDQYRNAS
NPLAHYDTTADEILQQCDGKLDMLVASVGTGGTITGIARKLKEKCPGCR
IIGVDPEGSILAEPEELNQTEQTTYEVEGIGYDFIPTVLDRTVVDKWFK
SNDEEAFTFARMLIAQEGLLCGGSAGSTVAVAVKAAQELQEGQRCVVIL
PDSVRNYMTKFLSDRWMLQKG
(SEQ ID NO: 33)
HELIGHTPIVEITRFSLPEGVRLFAKLEFYNPGGSVKDRLGRELIEDAL
EKGLVTEGGTIIEPTAGNTGIGLALAALQHDLRVIVCVPEKFSIEKQEL
MKALGATVVHTPTEQGMTGAIAKAKELVNEIPNSYSPSQFANEANPRAY
FKTLGPELWSALNGEINIFVAGAGTGGTFMGTASYLKEKNIDIKTVIVE
PEGSILNGGKAGSHETEGIGLEFIPPFLKTSYFDEIHTISDRNAFLRVK
ELAQKEGLLVGSSSGAAFHASLLEAEKAAPGTNIVTIFPDSSERYLSKD
IYKGWE
(SEQ ID NO: 34)
LDLIGNTPLVRVTRFDTGPCTLYLKLESQNPGGSIKDRIGVAMIEAAER
DGRLRPGGTIVEATAGNTGLGLALVGRAKGYRVVLVVPDKMSTEKVLHL
RAMGAEVHITRSDVGKGHPEYYQDVAARLAQDIPGAFFADQFNNPANPL
AHECGTGPELWAQTGHDLDAIVVGVGSSGTLTGLTRFFQKVQPELEMVL
ADPEGSIMAEYSRSGTLGTPGSWAVEGIGEDFVPAIADLSSVRHAYSIS
DEESFAMARELLRVEGIPGGSSTGTLLAAALRFCREQKEPKRVVSFVCD
TGTRYLSKIYNDQWMTDQG
(SEQ ID NO: 35)
YDLIGNTPLVLLEHYSDDKVKIYAKLEQWNPGGSVKDRLGKYLVEKAIQ
EGRVRAGQTIVEATAGNTGIGLAIAANRHHLKCKIFAPYGFSEEKINIM
IALGADVSRTSQSEGMHGAQLAARSYAEKYGAVYMNQFESEHNPDTYFH
TLGPELTSALQQIDYFVAGIGSGGTFTGTARYLKQHHVQCYAVEPEGSV
LNGGPAHAHDTEGIGSEKWPIFLERRLVDGIFTIKDQDAFRNVKSLAIN
EGLLVGSSSGAALQGALNLKAQLSEGTIVVVFPDGSDRYMSKQIFNYEE
NDYE
(SEQ ID NO: 36)
MQEKDASSQGFLPHFQHFATQAIHVGQDPEQWTSRAVVPPISLSTTFKQ
GAPGQHSGFEYSRSGNPTRNCLEKAVAALDGAKYCLAFASGLAATVTIT
HLLKAGDQIICMDDVYGGTNRYFRQVASEFGLKISFVDCSKIKLLEAAI
TPETKLVWIETPTNPTQKVIDIEGCAHIVHKHGDIILVVDNTFMSPYFQ
RPLALGADISMYSATKYMNGHSDVVMGLVSVNCESLHNRLRFLQNSLGA
VPSPIDCYLCNRGLKTLHVRMEKHFKNGMAVAQFLESNPWVEKVIYPGL
PSHPQHELVKRQCTGCTGMVTFYIKGTLQHAEIFLKNLKLFTLAESLGG
FESLAELPAIMTHASVLKNDRDVLGISDTLIRLSVGLEDEEDLLEDLDQ
ALKAA
(SEQ ID NO: 37)
MRAKTKLIHGIRIGEPSTGSVNVPIYQTSTYKQEAVGKHQGYEYSRTGN
PTRAALEEMIAVLENGHAGFAFGSGMAAITATIMLFSKGDHVILTDDVY
GGTYRVITKVLNRFGIEHTFVDTTNLEEVEEAIRPNTKAIYVETPTNPL
LKITDIKKISTLAKEKGLLTIIDNTFMTPYWQSPISLGADIVLHSATKY
LGGHSDVVAGLVVVNSPQLAEDLHFVQNSTGGILGPQDSFLLLRGLKTL
GIRMEEHETNSRAIAEFLNNHPKVNKVYYPGLASHQNHELATEQANGFG
AIISFDVDSEETLNKVLERLQYFTLAESLGAVESLISIPSQMTHASIPA
DRRKELGITDTLIRISVGIEDGEDLIEDLAQALA
(SEQ ID NO: 38)
MSQHDQHPDAPAQAFATRVIHAGQAPDPSTGAIMPPIYANSTYIQESPG
VHKGLDYGRSHNPTRWALERCVADLEGGTQAFAFASGLAAISSVLELLD
AGSHIVSGNDLYGGTFRLFERVRRRSAGHRFSFVDPTDLQAFEAALTPE
TRMVWVETPSNPLLRLTDLRAIAQLCRARGIISVADNTFASPYIQRPLE
LGFDVVVHSTTKYLNGHSDVIGGIAIVGDNPDLRERLGFLQNSVGAISG
PFDAFLTLRGVKTLALRMERHCSNALALAQWLERQPQVARVYYPGLASH
PQHELAKRQMRGFGGMISLDLRCDLAGARRFLENVRIFSLAESLGGVES
LIEHPAIMTHASIPAETRADLGIGDSLIRLSVGVEALEDLQADLAQALA
KI
(SEQ ID NO: 39)
MNKKTKLIHGGHTTDDYTGAVTTPIYQTSTYLQDDIGDLRQGYEYSRTA
NPTRSSVESVIAALENGKHGFAFSSGVAAISAVVMLLDKGDHIILNSDV
YGGTYRALTKVFTRFGIEVDFVDTTHTDSIVQAIRPTTKMLFIETPSNP
LLRVTDIKKSAEIAKEHGLISVVDNTFMTPYYQNPLDLGIDIVLHSATK
YLGGHSDVVAGLVATSDDKLAERLAFISNSTGGILGPQDSYLLVRGIKT
LGLRMEQINRSVIEIIKMLQAHPAVQQVFHPSIESHLNHDVHMAQADGH
TGVIAFEVKNTESAKQLIKATSYYTLAESLGAVESLISVPALMTHASIP
ADIRAKEGITDGLVRISVGIEDTEDLVDDLKQALDTL

1. A method for treating a subject having a microbial infection, said method comprising administering to said subject a therapeutically effective amount of at least one inhibitor of endogenous H₂S production by an organism causing the microbial infection.

2. The method of claim 1, further comprising administering a second antimicrobial compound.

3. The method of claim 1, wherein the inhibitor of endogenous H₂S production inhibits an H₂S-generating enzyme within the organism causing the microbial infection.

4. The method of claim 3, wherein the inhibitor of endogenous H₂S production inhibits a microbial enzyme selected from the group consisting of cystathionine β-synthase (CBS), cystathionine γ-lyase (CSE), 3-mercaptopyruvate sulfurtransferase (3MST), cystathionine γ-lyase (CSE), and paralogs thereof.

5-7. (canceled)

8. The method of claim 1, wherein the inhibitor of endogenous H₂S production is selected from the group consisting of amino-oxyacetate (AOAA), hydroxylamine, D,L-propargylglycine (PAG), β-cyano-L-alanine, aspartate and a derivative thereof, and homocysteine.

9-19. (canceled)

20. The method of claim 2, wherein said second antimicrobial compound is selected from the group consisting of a quinolone, an acridine, a phenothiazine, an aminoglycoside, a macrolide, an amphenicol, a steroid, an ansamycin, an antifolate, a polymyxin, a glycopeptide, a cephalosporin, a lactam, and any combination thereof.

21-26. (canceled)

27. The method of claim 1, further comprising administering an inhibitor of endogenous microbial nitric oxide (NO) production or a NO scavenger.

28-35. (canceled)

36. The method of claim 1, wherein said inhibitor of endogenous H₂S production selectively inhibits a H₂S-generating enzyme within the organism causing the microbial infection, but not a H₂S-generating enzyme in the cells of the subject being treated.

37. A method for enhancing efficacy of an antimicrobial treatment in a subject having a microbial infection, wherein said antimicrobial treatment comprises administering to the subject a first antimicrobial compound that becomes compromised by H₂S or natural products of H₂S metabolism in vivo, said method comprising co-administering said first compound with a therapeutically effective amount of a second compound which second compound is an inhibitor of endogenous H₂S production by an organism causing the microbial infection.

38-41. (canceled)

42. The method of claim 37, wherein the inhibitor of endogenous H₂S production inhibits an H₂S-generating enzyme within the organism causing the microbial infection.

43. The method of claim 42, wherein the inhibitor of endogenous H₂S production inhibits a microbial enzyme selected from the group consisting of cystathionine β-synthase (CBS), cystathionine γ-lyase (CSE), 3-mercaptopyruvate sulfurtransferase (3MST), cystathionine γ-lyase (CSE), and paralogs thereof.

44-46. (canceled)

47. The method of claim 37, wherein the inhibitor of endogenous H₂S production is selected from the group consisting of amino-oxyacetate (AOAA), hydroxylamine, D,L-propargylglycine (PAG), β-cyano-L-alanine, aspartate and a derivative thereof, and homocysteine.

48-58. (canceled)

59. The method of claim 37, wherein the first compound is selected from the group consisting of a quinolone, an acridine, a phenothiazine, an aminoglycoside, a macrolide, an amphenicol, a steroid, an ansamycin, an antifolate, a polymyxin, a glycopeptide, a cephalosporin, a lactam, and any combination thereof.

60-61. (canceled)

62. The method of claim 37, further comprising administering an inhibitor of endogenous microbial nitric oxide (NO) production or a NO scavenger.

63-70. (canceled)

71. The method of claim 37, wherein said inhibitor of endogenous H₂S production selectively inhibits a H₂S-generating enzyme within the organism causing the microbial infection, but not a H₂S-generating enzyme in the cells of the subject being treated.

72. A method for sensitizing a microbial pathogen to oxidative damage comprising administering to said pathogen an effective amount of at least one inhibitor of endogenous H₂S production by said pathogen.

73. The method of claim 72, wherein said pathogen is in a subject and the inhibitor of endogenous H₂S production is administered to the subject.

74-75. (canceled)

76. The method of claim 72, wherein the inhibitor of endogenous H₂S production inhibits an H₂S-generating enzyme within said pathogen.

77-90. (canceled)

91. The method of claim 72, further comprising administering an inhibitor of endogenous microbial nitric oxide (NO) production or a NO scavenger.

92-99. (canceled)

100. The method of claim 72, wherein said inhibitor of endogenous H₂S production selectively inhibits a H₂S-generating enzyme within said pathogen, but not a H₂S-generating enzyme in the cells of the subject being treated.

101. A pharmaceutical composition comprising (i) an antimicrobial compound that becomes compromised by H₂S or natural products of H₂S metabolism in vivo and (ii) an inhibitor of microbial endogenous H₂S production.

102. The composition of claim 101 further comprising (iii) an inhibitor of microbial endogenous nitric oxide (NO) production or a NO scavenger.

103. A pharmaceutical composition comprising (i) an inhibitor of microbial endogenous H₂S production and (ii) an inhibitor of microbial endogenous nitric oxide (NO) production or a NO scavenger.

104-121. (canceled)
